(19) United States
(12) Patent Application Publication
(10) Pub. No.: US 2003/0096976 A1
(43) Pub. Date: May 22, 2003

(54) Humanized antibodies LB-00503 and LB-00506 specific for human 4-1BB and pharmaceutical compositions comprising said humanized antibodies

(76) Inventors: Hyo Jeong Hong, Taejon (KR); Sung Sup Park, Taejon (KR); Young Jun Kang, Daejon (KR); Chang-Yuil Kang, Seoul (KR); Sung Kwan Yoon, Taejon (KR); Youngwoo Park, Daejon (KR); Hyesung Yoon, Daejon (KR); Hyunsook Jang, Seoul (KR); Geun Bae Rha, Daejon (KR); Jin-San Yoo, Daejon (KR); Jong Keun Jeong, Seoul (KR); Dong Sup Shim, Daejon (KR); Mijeong Park, Daejon (KR); Hwadong Kim, Kyung-gi Province (KR); Jung-Gyu Park, Seoul (KR); Jae-Young Yang, Daejon (KR)

Correspondence Address: BIRCH STEWART KOLASCH & BIRCH, PO BOX 747, FALLS CHURCH, VA, 22040-0747, US

(21) Appl. No.: 10/233,996

(22) Filed: Sep. 4, 2002

Related U.S. Application Data
(63) Continuation-in-part of application No. 09/438,954, filed on Nov. 12, 1999, now Pat. No. 6,458,934.

(30) Foreign Application Priority Data
May 11, 1999 (KR) 1999-0016750
Nov. 17, 1998 (KR) 1998-49177

(51) Int. Cl. A61K 39/395; C12N 5/06; C07K 16/44

(52) U.S. Cl. 530/388.15; 424/141.1; 435/328; 435/320.1

(57) ABSTRACT

The present invention relates to humanized monoclonal antibodies LB-00503 and LB-00506, which are specific for human 4-1BB molecules, have high binding affinities and can bind efficiently with activated T cells expressing the 4-1BB molecule, as well as pharmaceutical compositions. Particularly, the present invention provides said humanized antibody LB-00503, which is modified from the humanized antibody Hz4B4-2 and substitutes the 61st amino acid, serine by asparagine in the amino acid residues of 59th˜61st and said humanized antibody LB-00506, which enhances the antibody binding affinity of said humanized antibody LB-00503 in which 2 amino acid residues of the right border in the antibody binding site CDR2 of the heavy chain variable region are substituted from glutamine (Q)—glycine (G) to lysine (K)—serine (S).

### FIELD OF THE INVENTION

The invention relates to humanized antibodies that specifically bind to 4-1BB receptor proteins, preferably to human 4-1BB receptor protein. The humanized antibodies can be used as diagnostic reagents or can be formulated into pharmaceutical compositions for administration to a patient.

In particular, the present invention relates to humanized antibodies and pharmaceutical compositions comprising humanized antibodies. More particularly, the present invention relates to humanized monoclonal antibodies LB-00503 and LB-00506 which are specific for human 4-1BB molecule, have high binding affinities, can bind to activated T cells expressing the 4-1BB molecule effectively and are similar to human antibodies structurally and to pharmaceutical compositions comprising said humanized monoclonal antibodies.

### BACKGROUND OF THE INVENTION

The immune system has tremendous diversity and because the repertoire of specificities expressed by the B- and T-cell populations is generated randomly, it is bound to include many which are specific for self components. Thus, the body must establish self-tolerance mechanisms to distinguish between self and non-self determinants so as to avoid autoreactivity. However, all mechanisms have a risk of breakdown. The self-recognition mechanisms are no exception, and a number of diseases have been identified in which there is autoimmunity due to copious production of autoantibodies and autoreactive T cells.

Presently, more than 30 diseases are known to be caused by autoimmune responses or related to autoimmune reactions in their pathological progress. For example, as major cases of the diseases, rheumatoid arthritis, inflammatory bowel disease, systemic erythematous lupus, myasthenia gravis, pemphigus vulgaris, glomerulonephritis, pernicious anemia, thyroiditis and systemic lupus erythematosus, glomerular nephritis, malignant aplastic anemia, thyroid gland disease, testitis and the like are included. In Korea, rheumatoid arthritis has a high frequency of invasion and is provoked in one person per the population of 100 persons.

In various autoimmune diseases, functional cells such as B lymphocytes may be enhanced, basically. Otherwise, the functional lesion in the immune regulation of T lymphocyte and the hyper sensitization of B lymphocyte may work together.

The transplantation of tissues to replace diseased organs is now an important medical therapy. In most cases, adaptive immune responses to the grafted tissues are the major impediment to successful transplantation. When tissues containing nucl response against the grafted organ. Matching the MHC type of donor and genetic differences at other loci still trigger rejection.

Nowadays, the transplantation has been accomplished actively by use of various organs. In the course of graft operations, an immuno-suppressant should be applied. During the period of 1960 to early 1980, prednisone and azathioprine as therapeutic agents for immuno-suppression were administered. Then, in 1983, the FDA granted the use of cyclosporine for organ transplantation in the U.S.A. and new immuno-suppressive therapy was finally started. Recently, new immuno-suppressants, such as anti-lymphocyte globulin, anti-thymocyte globulin, OKT3 and the like also have been developed. Therefore, four kinds of immuno-suppressant are utilized in order to prevent and to treat the rejection induced after organ transplantation. Unfortunately, the above immuno-suppressants cause inevitable and serious side effects since they also affect normal immunocytes and other types of cells except immunocyte. Hence, it is necessary to develop new immuno-suppressants, which can be specific for the activated immunocytes, have an excellent efficacy to suppress the immune reaction and have no side effects.

The enhanced immune reaction in T lymphocytes or B lymphocytes can be suppressed by regulation theoretically. In clinical fields, methotrexate and cytotoxic agents, irradiation, thoracic duct drainage, anti-lymphocyte serum such as anti-lymphocyte globulin (ALG) and anti-thymocyte globulin (ATG) and other immuno-suppressants are utilized. Recently, some advanced countries, especially in the pharmaceutical industry, have progressed the various stages of clinical trials, in which monoclonal antibody-modifying chimera and humanized antibodies are adopted for application.

The immune system may also be manipulated or controlled to suppress unwanted immune responses in autoimmune disease and graft rejection. Currently, several different immunosuppressive agents have been used clinically. The examples are methotrexate, azathiopurine, cyclophosphamide, prednisone, cyclosporine A, FK506 (tacrolimus), anti lymphocyte globulin (ALG) and anti-thymocyte globulin (ATG). Very recently, antibodies by virtue of their exquisite specificity, have been utilized for the therapeutic inhibition of specific immune responses. The target molecules for these antibodies can be divided into two groups. The first group includes molecules that are expressed on the surface of lymphocytes, such as CD3, CD4, IL-2R, CDw52 and ICAM-1. The other groups are mainly cytokines such as TNF-α and IL-6. Some of the antibodies are effective and are being sold as pharmaceutical products.

However, the presently developed immunosuppressants have a common problem in that cells that are not related to immune response or normal cells are all affected by the drugs. This causes serious side effects that cannot be avoided. Therefore, an immunosuppressant is desired that is specific for activated immune cells, has excellent immunosuppressive activity, and has no adv

Although murine monoclonal antibodies are extensively used as diagnostic agents, their utility as therapeutics has been proven in only a few cases. The limited application is attributed to three major reasons. First, the repeated administration of murine monoclonal antibodies to humans usually elicits human immune responses against these molecules. The human anti-mouse antibody (HAMA) responses are directed to two different domains. The responses against the variable region are so called anti-idiotype responses which could block the antigen binding activity of murine antibodies. The responses against the constant region represent anti-isotype responses, which block the effector function of antibodies. The HAMA responses not only block the functions of newly administered antibodies but also result in formation of immune complexes with the murine antibodies, which cause some side effects and could reduce the half life of the antibody. Second, the half-life of murine antibodies even in the absence of immune complex formation is much shorter than that of human antibodies in vivo. Third, the effector functions through the Fc region of murine antibodies are weak or non-existent compared to those of human antibodies. All of the factors described above reduce the efficacy of murine monoclonal antibodies and are common problems related to human immunotherapy based on xenogeneically derived monoclonal antibodies.

To overcome the intrinsic undesirable properties of murine monoclonal antibodies, recombinant murine antibodies engineered to incorporate regions of human antibodies, so called “humanized antibodies”, have been developed. This alternative strategy was adopted as is was very difficult to generate human antibodies directed to human antigens, such as cell surface molecules, due to tolerance of the immune system against self-antigens. A humanized antibody contains complementarity determining region (CDR) regions and a few other amino acids of a murine antibody and the rest of the structure is derived from a human antibody.

### SUMMARY OF THE INVENTION

4-1BB is expressed on the surface of activated T-cells as a type o related to tumor necrosis factor receptor (TNFR) (Malett et al., Immunol. Today 12:220 (1991)). 4-1BB has a molecular weight of 55 kDa, and is found as a homodimer. In addition, 4-1BB binds to the protein kinase p₅₆^{lck} inside the cell. It has been suggested that 4-1BB mediates a signal transduction pathway from outside of the cell to inside (Kim et al., J. Immunol. 151:1255 (1993)). In this study, a portion of the 4-1BB molecule was observed to be on the interior side of cells and to associate with p561ck protein, a kind of protein kinase. Therefore, it is assumed that extracellular signals may be transferred into cells through the 4-1BB molecule.

A human 4-1BB gene was isolated from a cDNA library made from activated human peripheral T-cell mRNA (Goodwin et al., Eur. J. Immunol. 23:2631 (1993)). In this study, 4-1BB molecules were reported to be accessory molecules expressed on the surface of T cells and antigenic cells. It was previously found that the amino acid sequence of human 4-1BB shows 60% homology to mouse 4-1BB (Kwon et al., Proc. Natl. Acad. Sci. USA 86:1963(1989)), which indicates that the sequences are highly conserved. Especially, the homology demonstrates higher conservation in the cytoplasmic region. Moreover, in this study, 4-1BB molecules were shown to be homodimers with a molecular weight of 55 kDa and produced actively in various types of cells such as T cell line activated with ConA, phytohemaglutinin (PHA), ionomycin, anti-CD3 or the like, thymocyte, mature T cell and so on. (See also Pollock, et al., J. Immunol., 150, 771-781, (1993)). Moreover, analysis of the amino acid sequence of 4-1BB indicates that it belongs to the nerve growth factor superfamily, along with CD40, CD27, TNFR-I, TNFR-II, Fas, and CD30 (Alderson et al., Eur. J. Immunol. 24:2219 (1994). When a monoclonal antibody is bound to 4-1BB expressed on the surface of mouse T-cells, anti-CD3 T-cell activation is increased many fold (Pollok et al., J. Immunol. 150:771 (1993)).

4-1BB binds to a high affinity ligand (4-1BBL) expressed on several antigen-presenting cells such as macrophages and activated B cells (Pollok et al., J. Immunol. 150:771 (1993); Schwarz and their proliferation (Goodwin et al., Eur. J. Immunol. 23:2631 (1993); Alderson et al., Eur. J. Immunol. 24:2219 (1994); Hurtado et al., J. Immunol. 155:3360 (1995); Pollock et al. regulation of T cell-mediated immune responses (Ignacio et al., Nature Med. 3:682 (1997)).

The inventors have previously constructed a hybridoma producing a mouse monoclonal antibody that is specific for human 4-1BB (h4-1BB) expressed on the surface of activated T-cells (Korean patent laid-open no. 96-37064) and a hybridoma cell line producing the same (Korean Patent Registration No. 10-204733-0000). As a result of the search by the inventors for an immunosuppressant specific for activated T-lymphocytes and that also has no adverse side effect, the inventors have constructed a humanized monoclonal antibody from a mouse monoclonal antibody to 4-1BB that is expressed only in activated T lymphocytes (Korean patent laid-open no. 96-37064). The humanized monoclonal antibody has high affinity for 4-1BB. Administering the humanized anti-4-1BB monoclonal antibody to non-human primates does not elicit an anti-antibody response. Rather, it elicits strong immunosuppressive activity.

In addition, the present inventors have prepared humanized antibodies Hz4B4-1 and Hz4B4-2 which are modified to humanize the above mouse monoclonal antibody (Korean Laid-open Patent Publication No. 10-2000-034847). Concretely, the humanized antibody Hz4B4-1 is less humanized to magnify the binding affinity for a human 4-1BB molecule and the humanized antibody Hz4B4-2 is humanized more actively than the humanized antibody Hz4B4-1.

However, the inventors also have found that the humanized antibody Hz4B4-2 reduces the binding strength toward activated T cells remarkably and thus is difficult to be commercialized.

In order to overcome the foregoing and other disadvantages of the humanized antibody, the inventors of the present invention have tried to develop new humanized monoclonal antibodies which magnify the degree of humanization and coincidently improve the binding affinity for T cells.

Thus, one object of the present invention is to provide a humanized monoclonal antibody that specifically binds 4-1BB, especially human 4-1BB (h4-1BB) that has a high affinity for 4-1BB (h4-1BB). The humanized antibodies of the present invention have high affinity for human 4-1BB, and bear sequence similarity to human antibodies. Because the 4-1BB receptor protein that is specifically bound by the antibodies of the invention appears to be involved in activation of the immune response, the product can be used effectively to treat autoimmune diseases or it can be used as an immunosuppressant to prevent graft rejection. Because the antibodies of the present invention closely resemble human antibodies, they can be administered to a human patient without any negative side-effect, such as a human anti-mouse antibody response.

Another object of the present invention is to provide a pharmaceutical composition comprising the above humanized monoclonal antibodies, which can be applied to treat or suppress immune reactions for autoimmune diseases such as rheumatoid arthritis, inflammatory bowel disease and the like and for organ transplantation. The pharmaceutical composition is useful for treating autoimmune diseases or acting as an immunosuppressant to prevent graft rejection. As rheumatoid arthritis is thought to be caused by inappropriate activity of 4-1BB receptor, the composition is especially useful for treating rheumatoid arthritis.

Another object of the invention is to provide a diagnostic composition for diagnosis of immune dysfunctions related to over- or under-reactivity of 4-1BB receptor protein.

### BRIEF DESCRIPTION OF THE DRAWINGS

The above and other objects, features and other advantages of the present invention will be more clearly understood from the following detailed description taken in conjunction with the accompanying drawings, in which;

FIG. 1—Amino acid sequence comparison of the variable region of heavy (VH) and light chain (VL) of the humanized anti-human 4-1BB antibodies, Hz4B4-1 and Hz4B4-2. These sequences are compared with the amino acid sequence of mouse monoclonal antibody 4B4-1-1, human antibody VH M17750, and human antibody VL X82934.

FIG. 2—Location of the primers used in PCR synthesis of the genes encoding VH and VL of humanized antibody Hz4B4-1, and the location of humanization.

FIG. 3—Construction of the expression plasmid pRc-Hz4B4-k-gs and its restriction enzyme map.

FIG. 4—Construction of the expression plasmid pCI-Hz4B4-H and its restriction enzyme map.

FIG. 5—Location of the primer used in PCR synthesis of the genes encoding VH and VL of humanized antibody Hz4B4-2, and the location of humanization.

FIGS. 6a-6i—Change in the production level of OVA-specific IgG in baboons that have been immunized with ovalbumin and treated with humanized antibody Hz4B4-1.

FIGS. 7a-7d—FIGS. 7a and 7b show the proportion of cells reacting with 4B4 monoclonal antibody with CD4⁺ and CD8⁺ T-cells from the peripheral blood of normal people and rheumatoid arthritis patients. FIGS. 7c and 7d show the proportion of cells reacting with 4B4 monoclonal antibody with CD4⁺ and CD8⁺ T-cells from peripheral blood and synovial fluid of normal people and rheumatoid arthritis patients.

FIG. 8 depicts a schematic diagram of the humanization process to construct a heavy chain variable region of mouse monoclonal antibody 4B4-1-1 in the present invention.

FIG. 9 depicts a schematic diagram of the humanization process to construct a light chain variable region of mouse monoclonal antibody 4B4-1-1 in the present invention.

FIG. 10 depicts a comparison of amino acid sequences in the CDR2 site of the heavy chain variable region of humanized antibody.

FIG. 11 depicts an analysis of the humanized monoclonal antibodies Hz4B4-2, LB00503 and LB00506 by performing SDS-polyacrylamide gel electrophoresis.

FIG. 12 depicts an antibody-antigen binding affinity, which is measured by using the ELISA method.

FIG. 13 depicts the binding strength of the humanized antibody LB00506 toward activated T cells by using flow cytometry.

FIG. 14 depicts the standard one way mixed lymphocyte reaction by using the humanized monoclonal antibody LB00506.

### DETAILED DESCRIPTION OF THE PRESENT INVENTION

The present invention is embodied in two humanized monoclonal antibodies made from mouse monoclonal antibody 4B4-1-1 (Korean patent laid open no. 96-37064) that specifically binds human 4-1BB. The humanized monoclonal antibodies were used in human tests. The present invention also provides humanized monoclonal antibodies specific for human 4-1BB molecule, which improve the binding affinity toward human 4-1BB molecule and the binding strength to activated T cells excellently than the established humanized antibody Hz4B4-2.

The first humanized antibody Hz4B4-1 was made by grafting the antigen binding region, complementarity determining region (CDR), in the variable region of the mouse monoclonal antibody 4B4-1-1. In order to increase the antigen binding affinity of the humanized antibody, several amino acid residues were substituted in the framework region (FR) to resemble mouse antibody. The result is that the antibody has almost the same antigen binding affinity as the original mouse monoclonal antibody. The humanized antibody Hz4B4-1 is comprised of a light chain variable region having the amino acid sequence of SEQ ID NO:1 and a heavy chain variable region having the amino acid sequence of SEQ ID NO:2.

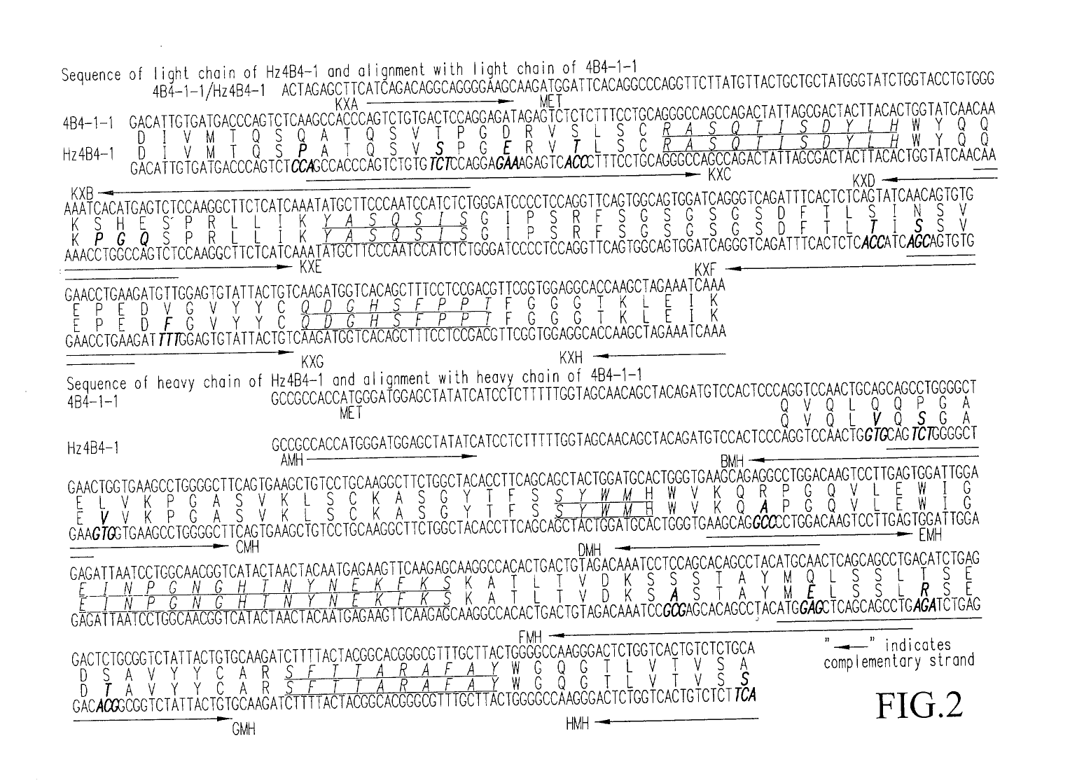

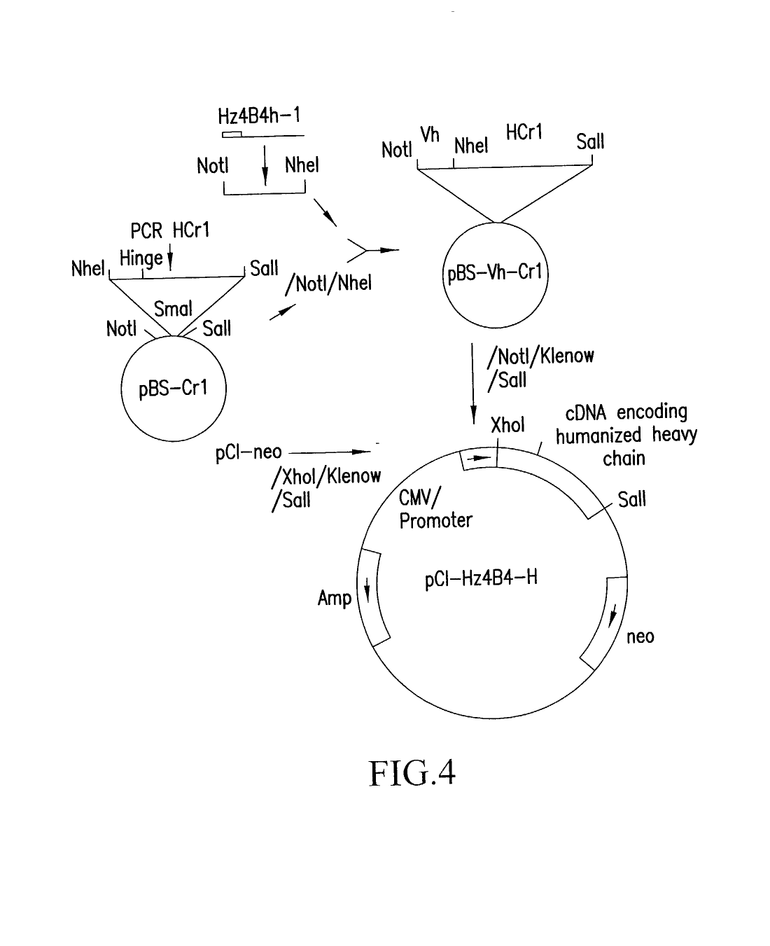

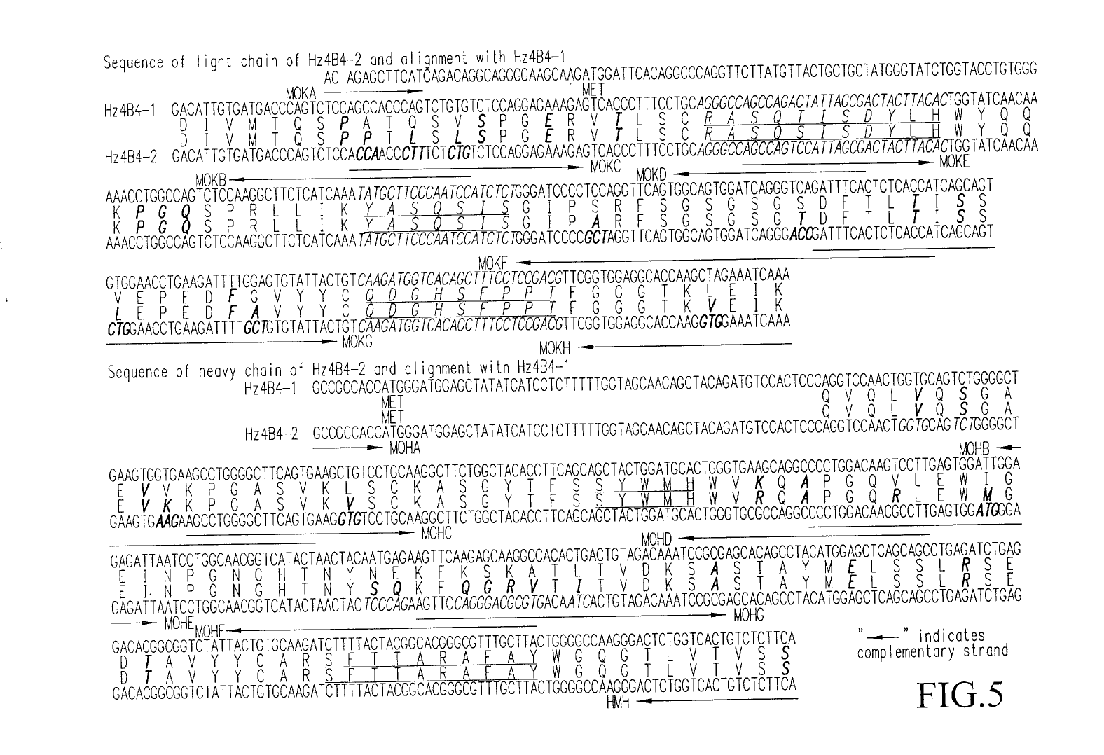

In order to make Hz4B4-1 more like a human antibody, additional amino acids in the mouse framework (FR) and complementarity determining regions (CDRs) (see, pp. 24-28 of Hood et al., “Immunology”, second ed. c. 1984 by Benjamin/Cumming Publishing Co., Inc., Menlo Park, Calif., esp. FIGS. 2-6 and 2-10; and pp. 288-296 of Paul, “Fundamental Immunology”, third ed. c. 1993 by Raven Press, Ltd., New York, N.Y., esp. Table 2) were substituted at several sites to resemble human antibody, and as a result the humanized antibody Hz4B4-2 was constructed. Hz4B4-2 has an antigen binding affinity that is 5 times greater than Hz4B4-1, and has 7 times higher antigen binding affinity than the original mouse monoclonal antibody. Humanized antibody Hz4B4-2 has a light chain variable region having an amino acid sequence shown in SEQ ID NO:3 and a heavy chain variable region having an amino acid sequence shown in SEQ ID NO:4.

The present inventors have already manufactured purified and examined the humanized monoclonal antibody Hz4B4-2. During the manufacturing process, the antibody Hz4B4-2 was observed to be glycosylated in its heavy chain and to be weakened remarkably in antigen binding affinity and in binding strength for activated T cells. Precisely, glycosylation within the variable region during the antibody modification not only affects the binding strength between antigen and antibody, but also makes it difficult to obtain homogeneous antibodies during purification. Therefore, a new humanized antibody which removes glycosylated sites and improves the antigen binding affinity and the binding strength to T cells outstandingly is required and tried to be developed.

Concretely, the humanized monoclonal antibody Hz4B4-2 contains amino acid residues with an asparagine (N)—tyrosine (Y)—serine (S) sequence in the 59th˜61st amino acid positions of the heavy chain, which is a feature of a typical glycosylated site (N-X-S). In order to remove the glycosylation site, the 61st amino acid, serine, is substituted by the asparagine of the mouse monoclonal antibody 4B4-1-1, a previous version of humanization. In this process, site directed mutagenesis was performed with the expression vector, pANTIBODY (KCTC 10125 BP) containing the gene of heavy chain and light chain of the humanized antibody Hz4B4-2 and the like are exploited. As a result, the modified antibody was obtained and named LB00503.

In addition, to improve the antigen binding affinity of the humanized monoclonal antibody LB00503, in the antigen binding site CDR2 of heavy chain variable region, two amino acid residues, glutamine (Q)—glycine (G) situated on the right border are substituted by the lysine (K)—serine (S) existing within the antigen binding site CDR2 which is the same as the mouse monoclonal antibody 4B4-1-1, a previous version of humanization. This modified antibody has been named LB00506. In detail, the above antibodies are identified so that glycosylation in the antibody Hz4B4-2 does not exist and the binding affinities to human 4-1BB molecule increases both in the antibodies LB00506 and LB00503, compared to that of antibody Hz4B4-2. Particularly, the binding affinity of the antibody LB00506 is identified to be comparable to that of the antibody Hz4B4-1.

The bacteria harboring the expression plasmid comprising the gene encoding the light chain Hz4B4-1 humanized antibody of the present invention, pRc-Hz4B4-Mok-gs, and the gene encoding the heavy chain, pCI-Hz4B4-MoH, were deposited at the Korean Institute of Science and Technology, Department of Life Sciences Institute Gene Bank, under accession numbers KCTC 0537BP and KCTC 0536BP, respectively, on Oct. 27, 1998.

The cell line MH200-3, producing the antibody Hz4B4-1 and SB500-23, producing Hz4B4-2 were deposited at the Korean Institute of Science and Technology, Department of Life Sciences Institute Gene Bank, under the accession numbers KCTC 0540BP and KCTC 0541BP, respectively, on Oct. 27, 1998.

Hz4B4-1 exhibits high affinity for human 4-1BB, as well as sequence similarity to human antibody. Hz4B4-1 can be used to treat autoimmune diseases and can be used as an effective immunosuppressant without encountering adverse side-effects.

LB00503 and LB00506 can also recognize a human 4-1BB molecule expressed on the surface of activated T cells and can be utilized for an immuno-suppressant so as to inhibit T cell activity by mediation of human 4-1BB molecules. Hence, LB00503 and LB00506 should bind better with 4-1BB molecules on the surface of activated T cells than a human 4-1BB molecule. In the meantime, the association between cell surface molecules and antibodies can be usually measured by performing flow cytometry with a FACS analysis instrument. Practically, the present inventors utilized a FACS analyzer to estimate the binding affinity between the humanized antibody LB00506 and the 4-1BB molecule on a cell surface and found that the binding affinity to antigen 4B4-1-1 is almost the same with that before humanization. Therefore, it is confirmed that the binding strength for activated T cells is not affected in the humanization process.

The antibody LB00506 also shows excellent immuno-suppression in the immune reaction by stimulated T cells.

Concretely, a standard one way mixed lymphocyte reaction (one way MLR) was performed to induce the immune reaction by mixing two kinds of peripheral blood mononuclear cells (hearinafter referred to as ‘PBMC’) obtained from the blood of genetically different persons. At that time, as an immune activator, one PBMC from one person is utilized for stimulating cells (stimulator) and the other PBMC from the other person for reacting cells. In this experiment, mitomycin C is treated before the stimulator and reactant cell are mixed for preventing the cell proliferation by itself. This procedure is a well-known method to identify the cell multiplication provoked by the immune reaction while T cells are mixed with other types of cells. Thus, it has a similar principle to the immuno-rejection, which is induced while operating organ transplantation. At that time, the cell proliferation can be estimated quantitatively by measuring the ³H-thymidine incorporation into DNA, which is an index of the increase in the cell number (DNA synthesis). In the present invention, the mixed lymphocyte reaction is utilized in order to identify whether anti-4-1BB humanized antibody LB00506 can inhibit the T cell proliferation effectively. Then, cyclosporin A (Sigma; 1 (g/ml) is adopted as a positive control substance and human IgG is selected as a negative control substance since it is not related with the 4-1BB molecule.

Consequently, both the humanized antibody LB00506 and Hz4B4-1 suppress the mixed lymphocyte reaction efficiently in 0.1, 1, and 10 g/ml concentrations. Therefore, the anti-4-1BB antibodies of the present invention, such as antibody LB00506 and Hz4B4-1, can be utilized as therapeutic agents for treating and inhibiting immuno-rejection induced in organ transplantation. In addition, it can be applied as an immuno-suppressant in various autoimmune diseases such as rheumatoid arthritis caused by excessive immune reaction and the like.

Meanwhile, the expression vector containing the gene of heavy chain and light chain in the humanized antibody Hz4B4-2 have been named pANTIBODY (accession number: KCTC 10152 BP), used as a template for in vitro mutagenesis, and deposited with the International Deposit Organization, the Korean Collection for Type Culture (KCTC) of the Korean Research Institute of Bioscience and Biotechnology (KRIBB) in Dec. 28, 2001. In addition, the cell line being transformed by pANTIBODY-VER6 and producing the humanized antibody LB00506 (accession number: KCTC BP) has been named B506-31-56 (CHO cell line) and deposited with the same organization on Jan. 31, 2002.

All of the humanized antibodies of the invention can be used as active ingredients in pharmaceutical compositions to treat autoimmune diseases, and can also be used as immunosuppressants. The pharmaceutical composition can be formulated as an oral or non-oral dosage form, for immediate or extended release. The composition can comprise inactive ingredients ordinarily used in pharmaceutical preparation such as diluents, fillers, disintegrants, sweeteners, lubricants and flavors. The pharmaceutical compositions of the present invention can contain additively and conventionally one or more substances such as flavoring agents, lubricants, solubilizers, suspending agents, filters, compression aids, binders or tablet-disintegrating agents.

The pharmaceutical compositions of the present invention can be prepared to be suitable for unit administration or for several administrations and can be administered orally or injected parenterally according to the purpose. The pharmaceutical composition is preferably formulated for intravenous administration, either by bolus injection or sustained drip, or for release from an implanted capsule. A typical formulation for intravenous administration utilizes physiological saline as a diluent. Typically, a human patient would ingest about 1 to 100 mg of the composition per kg of body weight once or several times.

It is to be understood that these dosage levels are only general guides and the proper dosage level for individual patients may vary considerably depending on factors such as antibody type, severity of disease, age, physical condition, sex, administration period, administration methods, body weight, diet, discharge etc.

The humanized antibodies of the present invention can also be utilized as therapeutic agents for autoimmune diseases or as an immuno-suppressant for inhibiting the immuno-rejection during organ transplantation, since they have high binding affinity to human 4-1BB molecules as well as homogeneous sequences of human amino acids. The present invention further provides pharmaceutical compositions comprising the above humanized monoclonal antibodies as an effective component, suitable carriers and so on. The pharmaceutical compositions of the present invention can be applied to treat or suppress immune reactions for autoimmune diseases such as rheumatoid arthritis, inflammatory bowel disease and the like and for organ transplantation.

Fab or Fab′ portions of the antibodies of the invention can also be utilized as the therapeutic active ingredient. Preparation of these antibody fragments is considered known in the art.

Formulation of antibodies for therapeutic administration is considered known in the art.

The dose for a patient population depends upon the specific antibody used, body weight, age, gender, state of health, diet, administration time and formulation of the composition, route of administration, and the disease to be treated. A typical dose is from 0.1 mg/kg/day to 100 mg/kg/day. More typically the dose is from 1 mg/kg/day to 50 mg/kg/day.

The composition of the present invention can also include printed matter that describes clinical indications for which the antibodies can be administered as a therapeutic agent, dosage amounts and schedules, and/or contraindications for administration of the antibodies of the invention to a patient.

The antibodies of the invention can also be used in a diagnostic assay. One preferred format for a diagnostic assay of the invention is quantification of cells in a sample that express h4-1BB on their surface. Methods for counting cells bearing particular surface markers are well-known in the art. For example, fluorescence activated cell sorting can be used. Another format for a diagnostic assay of the invention is to quantify the amount of h4-1BB protein in a sample. There are many formats for performing such an assay known in the art, for example antigen-immobilized or sandwich format enzyme-linked immunosorbent assays.

The invention is illustrated by the following Examples. The Examples are offered by way of illustration of the present invention, and not by way of limitation. Thus, it will be appreciated that those skilled in the art, on consideration of this disclosure, may make modifications and improvements within the spirit and scope of the present invention.

### EXAMPLE 1

### Design of the Humanized Antibody Hz4B4-1

In order to construct a humanized antibody, the amino acid sequences of the light chain and heavy chain variable regions of the mouse monoclonal antibody 4B4-1-1 (Korean laid-open application no. 96-37064) were compared with human sequences in the GenBank database. The human heavy chain variable region sequence M17750 (Dersimonian, H. et al., J. Immunol., 139, 2496 (1987)) having the greatest sequence similarity to the mouse 4B4-1-1 antibody heavy chain, and human light chain variable region sequence X82934 (Esposito, G. et al., Arch. Virol., 142, 601 (1997)) that has the most similarity to the mouse 4B4-1-1 antibody light chain were selected. In order to humanize the mouse monoclonal antibody 4B4-1-1, the CDR from the mouse antibody was grafted on to a human antibody. Also, 10 critical residues in the FR region of humanized light chain and 11 critical residues in the FR region of the humanized heavy chain were substituted with corresponding amino acids from the mouse 4B4-1-1 antibody.

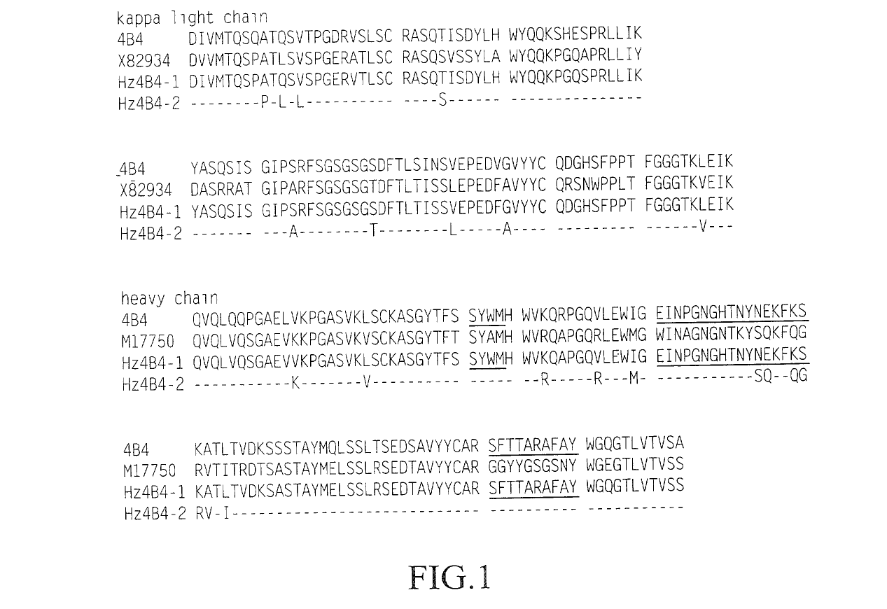

The humanized antibody Hz4B4k-1 light chain variable region designed as above and humanized heavy chain variable region Hz4B4h⁻¹ have the sequences designated SEQ ID NO:1 and SEQ ID NO:2, respectively. These sequences are compared with a light chain variable region sequence (SEQ ID NO:38) and a heavy chain variable region sequence (SEQ ID NO:39) of the mouse monoclonal antibody 4B4-1-1. These sequences are further compared to a light chain variable region sequence X82934 (SEQ ID NO:40) and a heavy chain variable region sequence M17750 (SEQ ID NO:41) of a human antibody. The alignment is shown in FIG. 1.

### EXAMPLE 2

### Construction of the Gene Encoding Humanized Antibody Hz4B4-1 and the Expression Plasmid

Primers were made that encompassed the base sequences in the regions in which the replacements were desired. These primers were KXA (SEQ ID NO:5), KXB (SEQ ID NO:6), KXC (SEQ ID NO:7), KXD (SEQ ID NO:8), KXE (SEQ ID NO:9), KXF (SEQ ID NO:10), KXG (SEQ ID NO:11), KXH (SEQ ID NO:12), AMH (SEQ ID NO:13), BMH (SEQ ID NO:14), CMH (SEQ ID NO:15), DMH (SEQ ID NO:16), EMH (SEQ ID NO:17), FMH (SEQ ID NO:18), GMH (SEQ ID NO:19), HMH (SEQ ID NO:20).

Among the above-described primers, the primers KXA (SEQ ID NO:5) to KXH (SEQ ID NO:12) were used to construct the gene encoding a humanized kappa light chain variable region. Primers AMH (SEQ ID NO:13) to HMH (SEQ ID NO:20) were used to construct the gene encoding a humanized heavy chain variable region. FIG. 2 shows by the location of the primers the regions of humanization in the humanized antibody Hz4B4-1, including the genes encoding the VL (SEQ ID NO:42), and the VH (SEQ ID NO:43). For comparison, FIG. 2 also shows the gene sequences of the light chain variable region (SEQ ID NO:44) and heavy chain variable region (SEQ ID NO:45) of the mouse monoclonal antibody 4B4 1-1.

The above primers were used to perform a polymerase chain reaction (PCR) using DNA encoding the light and heavy chain variable regions of the mouse monoclonal antibody to 4-1BB (Korean laid open patent no. 96-37064). The products were joined by an ordinary recombinant PCR method to form complete VL and VH cDNAs.

The PCR products of the humanized VL was cut with Hind III, then blunt ended with Klenow enzyme, and then cut again with Bgl II. The PCR product encoding VL that was made from primer HKD (SEQ ID NO:22) and primer Ryu-93 (SEQ ID NO:48), using the human VL (HCk) containing plasmid pAcS2-CK (Jin et al., Virus Research, 38:269-277 (1995)) as a template was inserted into a pBluescript™ plasmid. The so-constructed pBS-Ck plasmid was cut with SpeI, and blunt ended with Klenow enzyme, and then cut with BglII. Thus, the plasmid pBS-Vk-Ck that contains the humanized VL was constructed.

To insert the humanized VL into an expression vector, the EPO gene-containing HindIII-SalI fragment was removed from plasmid pcDNA-EPO-dEl-gs (Korean patent application no. 97-76923), and replaced with the humanized VL cDNA containing the NotI-SalI fragment from the plasmid pBS-Vk-Ck. The resultant plasmid is called pRc-Hz4B4-k-gs. Details of the construction of the expression plasmid pRc-Hz4B4-k-gs, and its restriction enzyme map are shown in FIG. 10.

To construct the heavy chain, similarly made PCR products of VH were cut with NotI and NheI. Then, the PCR product cDNA encoding the VH made from the plasmid pAcS2-CH (Jin et al., Virus Research, 38:269-277 (1995)), containing a human antibody VH gene, using primer HHCD (SEQ ID NO:21) and primer Ryu-101 (SEQ ID NO:49), was inserted into a pBluescript™ plasmid. The plasmid pBS-Crl resulted. PBS-Crl was digested with NotI and NheI, and the plasmid pBS-Vh-Crl was constructed. In order to insert the gene encoding the humanized heavy chain into an expression vector, pBS-Vh-Crl as digested with NotI, and blunt ended with Klenow, and then digested again with SalI, and then inserted into the XhoI-SalI site of the plasmid pCI-neo, which resulted in the plasmid pCI-Hz4B4-H. The details of the construction of the plasmid pCI-Hz4B4-H and its restriction enzyme map are shown in FIG. 11. The base sequence of the gene encoding the humanized light and heavy chains from each plasmid was confirmed by DNA sequence analysis. The humanized light and heavy chain genes in the expression plasmids were linked to a human cytomegalovirus (HCMV) promoter.

### EXAMPLE 3

### Expression of Humanized Antibody Hz4B4-1 and Selection of Cells

CHO-K1 cells (ATCC CCL61) cultured in GDMEM containing 10 dialyzed calf serum (FBS) in 5% CO₂ at 37° C., were inoculated into a 6 cm diameter dish so that 5×10⁵ cells were obtained. GDMEM contains DMEM (Gibco) and 4.5g/l glucose, 15 mg/l phenol red, 1 mM sodium pyruvate, 1.75 g/l sodium bicarbonate, 500 μM asparagine, 30 μM adenosine, 30 μM guanosine, 30 μM cytidine, 30 μM uridine, 10 μm thymidine, and non-essential amino acids (GIBCO). 2.5 μg of plasmid pRc-Hz4B4-k-gs and 2.5 μg of pCI-Hz4B4-H made in Example 2 were combined and diluted in 0.3 ml OPTI-MEM TM (GIBCO). Also, in 0.3 ml of OPTI-MEM I™, μl of lipofectamine™ (GIBCO) was diluted, mixed, and allowed to stand for 15 min.

The prepared CHO-K1 cells were washed 3 times in OPTI MEM I™. Next, the plasmid-lipofectamine™ mixture prepared as above was spread evenly over the cells. The cells were cultured in 5% CO₂ at 37° C. for 6 hr. Then, the culture medium was changed to 3 ml of GDMEM containing 10⁵ dialyzed calf serum, and the cells were cultured for an additional 48 hrs. In the culture medium, 3 ml of 0.25% trypsin (GIBCO) was added at 37° C. and allowed to react for 3 minutes, and centrifuged (1,000×g, 5 min.). The cells thus obtained were placed into 96-well plates at 2×10³ cells per well. After 48 hr, 5 μM methionine sulfoxamine (MSX) was added to the GDMEM containing 10% dialyzed calf serum and the cells were cultured in 5% CO₂ at 37° C. The culture medium was changed every 4 days; the culture was continued for 2 weeks.

The ability of each surviving cell clone to produce antibodies was assayed. An ELISA sandwich assay using goat anti-human IgG (Sigma) conjugated to horseradish peroxidase (HRP) (Park et al., Hybridoma, 15, 435-441 (1996)) was carried out to obtain clones producing antibodies. Among these, 5 clones (A6B, A9A, BlF, 212A, A7B) exhibited high production of antibodies. These 5 clones were placed in GDMEM containing 10% dialyzed calf serum. 100, 200, 350, 500 and 1000 μM MSX was added to each culture, and the cell clones that were most viable and that produced the most highest amount of antibodies were determined and separated out. The 3 clones that produced the largest amount of the product are shown in Table 1.

TABLE 1

| | Conc. of MSX | Production of |
|---|---|---|
| Clone | (μm) Ab | (μg/10⁶ cells/day) |
| A6B-200-2 | 200 | 11.3 |
| MH200-2 | 200 | 12.2 |
| MH200-3 | 200 | 16.2 |

### EXAMPLE 4

### Isolation and Purification of Humanized Antibody Hz4B4-1

The cells of the clone MH200-3 obtained in Example 3 were cultured in T175 flasks containing serum-free media (CHO-S-SFMII, GIBCO) in 5%CO₂ at 370 C. The culture conditioned medium was applied to a Protein G-Sepharose column (Pharmacia). The antibodies that bound to the column were eluted with on 10% SDS-PAGE. Bands of about 55 kDa (heavy chain) and about 25 kDa (light chain) were observed indicating that the humanized antibodies were purified.

### EXAMPLE 5

### Antigen-Binding Affinity of Humanized Antibody Hz4B4-1

The binding affinity of the mouse monoclonal antibody 4B4-1-1 and humanized antibody were determined and compared using the BIAcore™ assay (Pharmacia). Rabbit anti-mouse IgG (Sigma) and goat anti-human IgG (Fc-specific) were each diluted in 10 mM acetic acid buffer, and coupled to a Dextran CM-5 sensor chip (Pharmacia). 1 M ethanolamine was added to stop the reaction. Mouse monoclonal antibody 4B4-1-1 and humanized antibody Hz4B4-1 were each diluted to a concentration of 50 μg/ml in HEPES buffer (HBS). The coupled antibodies were bound at 100 resonance units (R.U.), and 25 μg/ml of 4-1BB antigen was applied at a flow rate of 10 μl/min. and bound for 5 minutes. HBS buffer (Pharmacia) was applied for 5 minutes at the same flow rate so that dissociation would occur. The association rate and dissociation rates and corresponding rate constants were determined by using BIA evaluation software. The results are shown in Table 2 as kₒₙ and k_{off} values; K_{d} is also shown.

TABLE 2
Antigen binding affinity of humanized antibody Hz4B4-1

| Antibody | kₒₙ (M⁻¹s⁻¹) | k_{off} (s⁻¹) | K_{d} (M) |
|---|---|---|---|
| Mouse | 1.16 × 10⁴ | 1.54 × 10⁻⁶ | 1.33 × 10⁻¹⁰ |
| monoclonal | | | |
| antibody 4B4 | | | |
| 1-1 | | | |
| humanized | 5.00 × 10⁴ | 4.36 × 10⁻⁶ | 8.72 × 10⁻¹¹ |
| antibody | | | |
| Hz4B4-1 | | | |

The results above show that the humanized antibody Hz4B4-1 has greater association and dissociation rate with the antigen compared with the mouse antibody. The antigen binding affinity (K_{d}) was about 1.5 times greater than the mouse antibody.

### EXAMPLE 6

### Design of the Humanized Antibody Hz4B4-2

In order to humanize HzHB4-1 even more, one amino acid residue in CDR1 of the light chain, and 8 amino acid residues in the mouse FR were replaced with corresponding human amino acid residues. Also, 4 amino acid residues were replaced in CDR2 of the humanized heavy chain, and 8 amino acid residues were replaced in the mouse FR with corresponding residues from a human antibody. As indicated above, the novel humanized light chain Hz4B4k-2 and humanized heavy chain Hz4B4h-2 have the amino acid sequence of SEQ ID NOS:3 and 4, respectively. These sequences were aligned with mouse monoclonal antibody 4B4-1-1 VL (SEQ ID NO:38) and VH (SEQ ID NO:39), human antibody VL X82934 (SEQ ID NO:40) and VH M17750 (SEQ ID NO:41), and Hz4B4-1 humanized antibody sequences (SEQ ID NOS:1 and 2). The alignment is shown in FIG. 8.

### EXAMPLE 7

### Construction of the Gene Encoding Humanized Antibody Hz4B4-2 and the Expression Plasmid

Primers were synthesized that encompassed the base sequences in the regions in which the substitutions were desired. These primers were MOKA (SEQ ID NO:23), MOKB (SEQ ID NO:24), MOKC (SEQ ID NO:25), MOKD (SEQ ID NO:26), MOKE (SEQ ID NO:27), MOKF (SEQ ID NO:28), MOKG (SEQ ID NO:29), MOKH (SEQ ID NO:30), MOHA (SEQ ID NO:31), MOHB (SEQ ID NO:32), MOHC (SEQ ID NO:33), MOHD (SEQ ID NO:34), MORE (SEQ ID NO:35), MOHF (SEQ ID NO:36), MOHG (SEQ ID NO:37).

Among the above primers, MOKA (SEQ ID NO:23) to MOKH (SEQ ID NO:30) were used to construct the gene encoding humanized kappa light chain variable region. Primers MOHA (SEQ ID NO:31) to MOHG (SEQ ID NO:37) were used to construct the gene encoding the humanized heavy chain variable region.

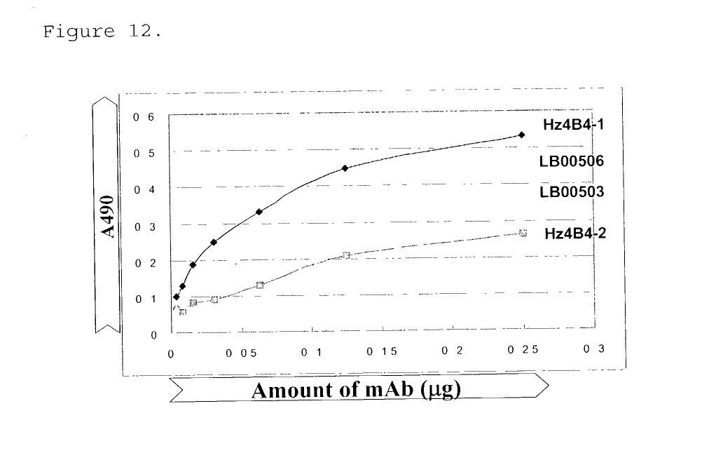

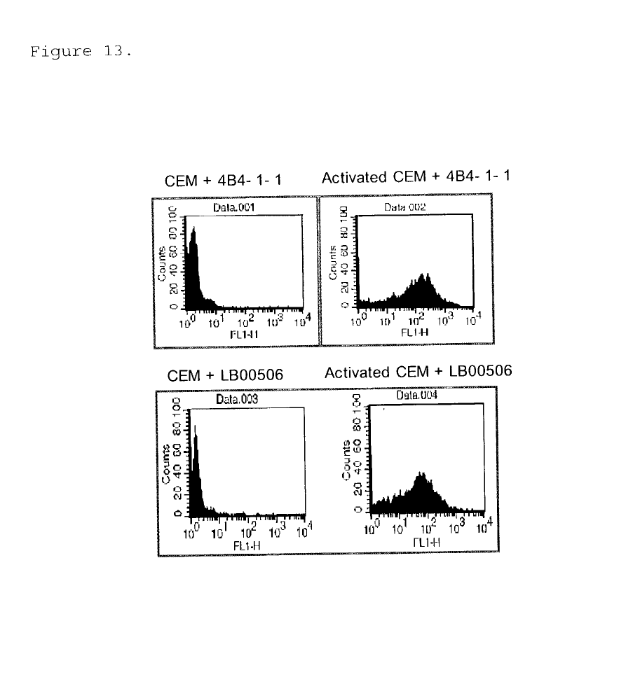

In addition, primer HMH (SEQ ID NO:20) was also used in the construction of the gene encoding humanized heavy chain variable region. FIG. 12 shows the humanized region of Hz4B4-1, and the VL gene (SEQ ID NO:42) and VH gene (SEQ ID NO:43) of Hz4B4-1.

The above MOK* series of primers were used with the light chain gene of the Hz4B4-l antibody as a template to produce a gene encoding the light chain of the humanized antibody for Hz4B4k-2. The above MOH* series of primers was used with the heavy chain gene of Hz4B4-l as a template to produce a VH gene and an ordinary recombinant PCR method was carried out to further humanize the antibody. The genes encoding VL (Hz4B4k-2, SEQ ID NO:46) and VH (Hz4B4h-2, SEQ ID NO:47) were synthesized. The Hz4B4k-2 DNA obtained above was cut with XbaI and BglII and inserted into the XbaI/BglII site of the Hz4B4-l light chain expression vector, pRC-Hz4B4-k-gs. Thus, the Hz4B4-2 light chain expression plasmid pRc-Hz4B4MoK-gs was constructed.

In the case of the heavy chain, the Hz4B4h-2 DNA was cut with XhoI and NheI, and inserted into the XhoI/NheI site of the heavy chain expression vector pCI-Hz4B4-H. Thus, the humanized antibody Hz4B4-2 heavy chain expression plasmid pCI-Hz4B4-MoH was constructed.

The base sequence of the gene encoding the humanized light and heavy chains from each plasmid was confirmed by DNA sequence analysis.

### EXAMPLE 8

### Expression of Humanized Antibody Hz4B4-2 and Selection of Cells

As in Example 3 above, CHO-K1 cells were transfected with pRc-Hz4B4-Mok-gs and pCI-Hz4B4-MoH, and the transformed cells were incubated for 2 weeks in GDMEM culture medium that contains 25 μM methionine sulphoximine (MSX), at 37° C., in 5% CO₂. Resistant clones were isolated, and antibody production was determined by sandwich ELISA. Among these clones, the high producing SB clones were cultured in GDMEM medium that contains 10% dialyzed calf serum, to which 100, 200, 350, 500 or 1000 μM MSX was added. The clones that were most viable and high antibody producers at 500 μM MSX were separated out. These clones produced about 3 μg/10⁶ cells/day of antibody.

### EXAMPLE 9

### Isolation and Purification of Humanized Antibody Hz4B4-2

As in Example 4, the above SB500 cells were cultured in serum-free medium. The conditioned culture medium was applied to a protein G-Sepharose (Pharmacia) affinity column. The purified antibodies were electrophoresed on 10% SDS-PAGE. Bands of about 55 kDa (heavy chain) and about 25 kDa (light chain) were observed, indicating that the humanized antibodies had been purified.

### EXAMPLE 10

### Antigen-Binding Affinity of Humanized Antibody Hz4B4-2

The antigen binding affinity of the purified Hz4B4-2 humanized antibody was measured as in Example 5 using the BIAcore™ assay (Pharmacia). The results are shown in Table 3.

TABLE 3
Antigen binding affinity of humanized antibody Hz4B4-2

| Antibody | kₒₙ (M⁻¹s⁻¹) | k_{off} (s⁻¹) | K_{d} (M) |
|---|---|---|---|
| Mouse | 1.16 × 10⁴ | 1.54 × 10¹⁶ | 1.33 × 10⁻¹⁰ |
| monoclonal | | | |
| antibody | | | |
| 4B4-1-1 | | | |
| humanized | 5.00 × 10⁴ | 4.36 × 10⁻⁶ | 8.72 × 10⁻¹¹ |
| antibody | | | |
| Hz4B4-1 | | | |
| humanized | 1.17 × 10⁴ | 2.14 × 10⁻⁶ | 1.83 × 10⁻¹¹ |
| antibody | | | |
| Hz4B4-2 | | | |

As shown in Table 3, Hz4B4-1 has 1.5 times greater antigen binding affinity (K_{d}) compared to mouse monoclonal antibody 4B4-1-1. Humanized antibody Hz4B4-2 has 7.3 times greater antigen binding affinity compared the mouse monoclional antibody. It is expected that the humanized antibodies Hz4B4-1 and Hz4B4-2 of the invention will have the same affinity in human beings.

### EXAMPLE 11

### Construction of the Humanized Antibody Hz4B4-1

Step 1: Comparison of mouse monoclonal antibody 4B4-1-1, humanized antibody Hz4B4-1 and humanized antibody Hz4B4-2

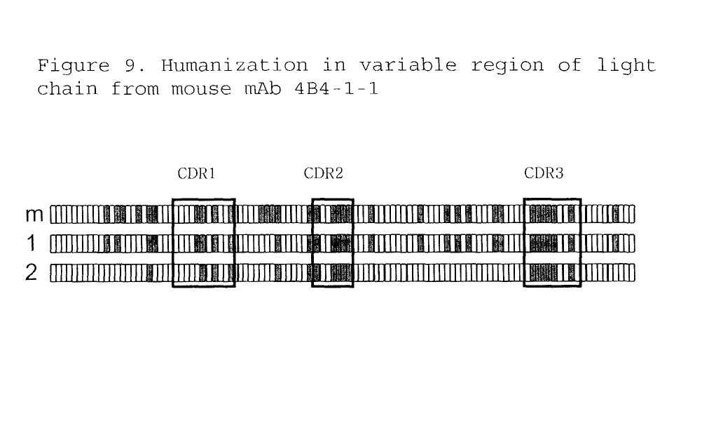

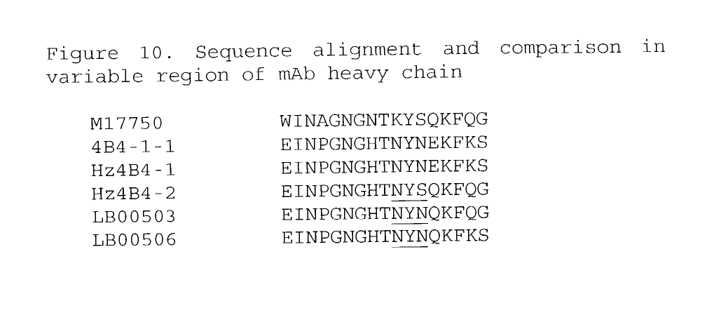

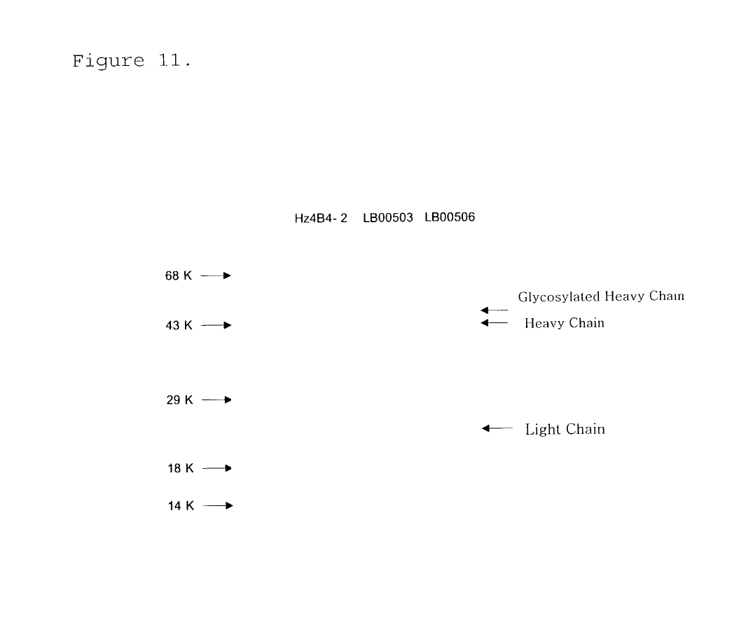

The inventors of the present invention have observed some disadvantages while developing the humanized antibody Hz4B4-2 for the commercialization. Concretely, the binding affinity toward activated T cells decreased remarkably. In order to solve the problem, mouse monoclonal antibody 4B4-1-1, humanized antibody Hz4B4-1 and humanized antibody Hz4B4-2 were examined and compared, respectively. The results are illustrated in FIG. 8 and FIG. 9.

FIG. 8 and FIG. 9 depict the humanization of mouse monoclonal antibody 4B4-1-1 homologous to human 4-1BB molecule schematically in the heavy chain variable region (108 amino acid residues) and in the light chain variable region (109 amino acid residues) respectively. In addition, the heavy chain gene M17750 (Dersimonian, H. et al., J. Immunol., 139, 2496-2501, 1987) and the light chain gene X82934 (Esposito, G. et al., Arch. Virol., 142, 601-610, 1997) for human antibody variable region were utilized in order to humanize the amino acid sequences of mouse monoclonal antibody 4B4-1-1 (Korean Laid-open Publication No. 1996-37064) in the heavy chain variable region and the light chain variable region independently. Then the amino acid sequences of mouse monoclonal antibody 4B4-1-1 and its humanized antibody were compared. Concretely, the amino acid residues of human antibody M17750 and X82934 are indicated with a white box and the specific amino acid residues of mouse monoclonal antibody 4B4-1-1 are depicted with a black box. Then, the important regions for binding with antigens were demonstrated with CDR1, CDR2 and CDR3. In FIG. 8 and FIG. 9, symbols, ‘m’, ‘1’ and ‘2’ denote the heavy chain variable region and the light chain variable region in the mouse monoclonal antibody 4B4-1-1, the humanized antibody Hz4B4-1 and the humanized antibody Hz4B4-2, respectively.

The inventors of the present invention have detected 2 kinds of problems while the amino acid sequences were compared in the heavy chain and the light chain as shown in FIG. 8 and FIG. 9. First, new glycosylation site (NYS) that did not previously exist was created on the CDR2 site of heavy chain in the case of the antibody Hz4B4-2 (See FIG. 10). Second, the CDR 2 right border of one heavy chain was substituted excessively by the amino acid residues of human antibody.

### EXAMPLE 12

### Construction and Examination of the Humanized Antibody LB00503 by Removing the Glycosylation Site of the Heavy Chain Variable Region in the Antibody Hz4B4-2

Step 1:

In order to investigate the glycosylation of the CDR2 site in the heavy chain variable region of the humanized monoclonal antibody Hz4B4-2, the antibody was purified and analyzed by performing 10% SDS-polyacrylamide gel electrophoresis (SDS-PAGE). As a result, the humanized antibody Hz4B4-1 was separated into the heavy chain with a molecular weight, of about 55 kDa and the light chain with a molecular weight of about 25 kDa as estimated previously. In the meantime, the humanized antibody Hz4B4-2 was depicted as double bands situated at about 55 kDa size of the heavy chain. At that time, the band corresponding to the heavy chain of antibody Hz4B4-1 was identified to be the same with the lower band observed in the antibody Hz4B4-2. Therefore, it is guessed that the heavy chain part of antibody Hz4B4-2 increases the molecular weight by the glycosylation (See FIG. 10).

Step 2: Removal of the Glycosylation Site by Using Site-Directed Mutagenesis

First of all, the humanized monoclonal antibody Hz4B4-2 contained amino acid residues asparagine (N)—tyrosine (Y)—serine (S) sequence at the 59th˜61st amino acids of the heavy chain, which was a typical feature of the glycosylation site (N-X-S). In order to remove the glycosylation site, the 61st amino acid, serine was substituted by the 61st amino acid, asparagine in the mouse monoclonal antibody 4B4-1-1, which was a previous version of the humanization by exploiting site directed mutagenesis. As a result, the modified antibody obtained was named LB00503.

In detail, the site-directed mutagenesis was performed by using the expression vector pANTIBODY, which was composed of genes encoding the light chain and the heavy chain of the humanized antibody HzB4-2 together and by exploiting the primer VER3 of SEQ ID NO: (5′-ACTAACTACAACCAGAAGTTC-3′). Then as an instrument for mutagenesis, the GeneEditor™ in vitro site-directed mutagenesis kit (Cat. #Q9280) purchased from Promega Inc. (Address: 2800 Woods Hollow Road, Madison, Wis., U.S.A.) was adopted and utilized according to the procedure recommended by the company. As a result, the expression vector constructed was named pANTIBODY-VER3.

Step 3: Preparation of the Cell Line Expressing the Antibody LB00503

In order to develop a cell line producing antibody protein stably, first DHFR negative CHO cell (ATCC CRL 9096) was diluted 12 times, allotted onto a 36 mm microplate in the previous day of transfection and then cultivated at 37(C in the CO₂ incubator. When the cells were examined to grow to about 70˜80%, they were transfected with the plasmid pANTIBODY-VER3 DNA by using Lipofectamine plus (Gibco BRL Cat. 10964-013/11514-015). At that time, IMDM medium (Isocove's modified Dulbecco's media; Gibco BRL Cat. 12200-069) containing 10% fetal bovine serum, 10 (g/ml of hypoxantine and 10 (g/ml of thymidine. After 2 days, the culture medium was replaced with complete alpha medium (MEM; minimum essential medium; Gibco BRL Cat. 12000-022) containing dialyzed in 800 g/ml fetal bovine serum with no hypoxanthin and no thymidine so that only transfected CHO cells can proliferate and be selected. After 10˜14 days progressed from the start of the selective cultivation, live cells formed colonies. Among these cells, good colonies that grow normally continued to be cultivated until their diameter and numbers reached 100 mm and 107 cells per culture plate respectively and then some of them were stored in the freeze-dried state and exploited to proceed to the next step of gene amplification.

In order to amplify the transformed gene obtained above, a DHFR inhibitor, methotrexate (MTX) was added to the selective medium. Then, the cells continued to be cultivated while the concentration of MTX was increased orderly so as to induce the antibody gene amplification with the DHFR gene. Concretely, the concentration of MTX started from 30 nM in the initial state and increased about 3 times in each respective stage. Then, in each concentration stage, more than 2˜3 rounds of successive cultivation were accomplished and the concentration of MTX was increased for the next stage when CHO cells proliferated normally and were maintained stably in the cell growth. Preferably, in 1 (M of MTX concentration, the cell line of the present invention was obtained due to a good state of cell growth.

Step 4: Purification and Analysis of Humanized Antibody LB00503

The cell line, which was prepared in the above Step 3 and expressed the humanized antibody LB00503 was cultivated by using CHO-S-SFMII medium (purchased from Gibco BRL) without serum in a T175 flask and maintained at 37(C, in 5% C02. Then the resulting culture medium was passed through Protein-G Sepharose column (Pharmacia), eluted to collect antibodies bound onto a column by using 0.1 M of glycine solution (pH 2.7), neutralized with 1 M of Tris-Cl buffer (pH 9.0) and dialyzed with PBS buffer (pH 7.0). Then the antibodies purified above were analyzed by performing 10% SDS-polyacrylamide gel electrophoresis (SDS-PAGE). As a result, the antibody was detected to be separated into a band of about 55 kDa which was the heavy chain and a band of about 25 kDa containing the light chain. Particularly, the 55 kDa band corresponding to the heavy chain was observed as a single band elucidating that double band which was shown in heavy chain region of Hz4B4-2 is caused by glycosylation on its variable region.

Step 5: Comparison of Antigen Binding Affinity in the Antibody LB00503 and the Antibody Hz4B4-2

In order to compare the antigen binding affinity of the humanized antibody LB00503 and the humanized antibody Hz4B4-2 purified above, GST-4-1BB was utilized as an antigen to perform the ELISA method (Park, et al., Hybridoma, 15, 435-441, 1996). The result is demonstrated in FIG. 12. Consequently, the humanized antibody LB00503 with the glycosylation site removed was measured to have a higher binding affinity for the antigen 4-1BB than the glycosylated antibody Hz4B4-2. Therefore, the glycosylation at the CDR2 site of the heavy chain variable region was identified to reduce the binding affinity towards the antigen 4-1BB.

### EXAMPLE 13

### Construction and Examination of the Humanized Antibody LB00506 with Higher Binding Affinity than that of the Humanized Antibody LB00503

As demonstrated above, the inventors of the present invention have found that the amino acid residues of the right border within CDR2 site were modified excessively and substituted from the heavy chain of the humanized antibody Hz4b4-2. In order to increase the antigen binding affinity of antibody LB00503, two amino acid residues, glutamine (Q)—glycine (G) within the antigen binding site CDR2 of the heavy chain variable region were substituted for lysine (K)—serine (S) within the same site of the mouse monoclonal antibody 4B4-1-1, which was a previous version of humanization by exploiting site directed mutagenesis. As a result, the modified antibody obtained was named LB00506.

Step 1: Site Directed Mutagenesis

In order to perform the mutagenesis, the expression vector pANTIBODY-VER3 was utilized as a template and the primer VER6 (5′-GAGAAGTTCAAGTCACGCGTGA CA-3′) was used as a primer. Then, the same procedure was accomplished as illustrated in Example 12. As a result, the modified plasmid has been named pANTIBODY-VER6.

Step 2: Preparation of the Cell Line Producing the Humanized Antibody LB00506

As illustrated in Example 12, the same procedure was performed in order to prepare the cell line (LB506-31-56 CHO cell line) of the present invention producing the humanized antibody LB00506 (KCTC 10165BP) and deposited with International Deposit Organization, the Korean Collection for Type Culture (KCTC) of the Korean Research Institute of Bioscience and Biotechnology (KRIBB)

Step 3: Purification and Examination of the Humanized Antibody LB00506

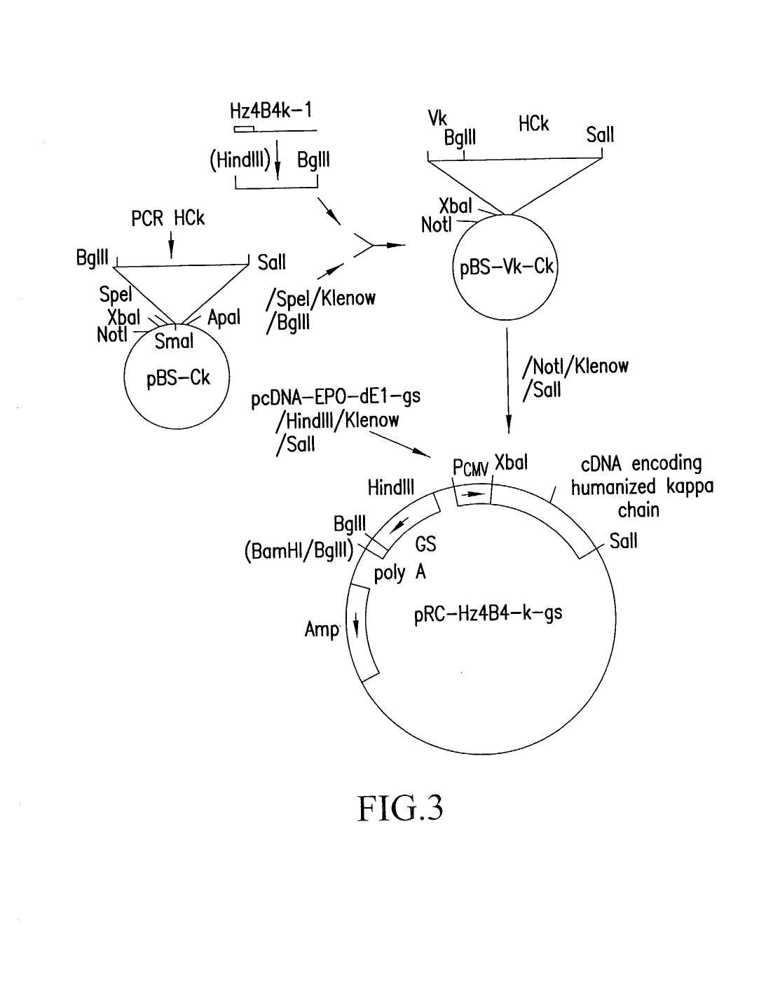

As illustrated in Example 12, the same procedure was performed in order to purify the humanized antibody LB00506. The purified antibody was separated with 10% SDS—polyacrylamide gel electrophoresis (SDS-PAGE) and analyzed. As a result, the antibody was examined to be separated into a band of about 55 kDa containing the heavy chain and a band of about 25 kDa containing light chain. Particularly, the 55 kDa band corresponding to the heavy chain was observed to be a single band (See FIG. 3).

Step 4: Analysis of the Antigen Binding Affinity in the Humanized Antibody LB00506

In order to measure the antigen binding affinities of the humanized antibody LB00506 and the antibody Hz4B4-2 purified above, the ELISA method was accomplished by using the antigen GST-4-2BB. The result depicted in FIG. 12. As a result, it was confirmed that the affinities toward human 4-1BB molecule are similar both in the humanized antibody LB00506 and the humanized antibody LB00503.

### EXAMPLE 14

### Examination of the Binding Affinity to Activated T Cells in the Humanized Antibody LB00506 by Using Flow Cytometry

Step 1: Activation of CEM T Cell

CEM cells were maintained in the range from 2˜3×10⁵ to 1˜2×10⁶ and culture medium was changed and added newly every 2 or 3 days. For the culture medium, RPMI 1640 containing 1.5 g/L sodium bicarbonate, 4.5 g/L glucose, 10 mM HEPES, 1.0 mM sodium pyruvate, 2 mM L-glutamine and 10% fetal bovine serum was utilized. In order to stimulate CEM cells, 1×10⁶ cells/ml were treated with 10 ng/ml of PMA and 1 (g of ionomycin and cultivated for 24 hours. Then the activated CEM cells were washed by using FACS washing buffer (PBS, 10 mM HEPES, 2% FBS, 0.1% sodium azide) and added with 1×10⁶ cells per sample. Herein, antibodies for the next tests were prepared by adjusting the cells to 50 (g/ml concentration respectively and 100 (1 was added so as to be reacted at 4(C for 40 minutes. Then, the resulting cells were washed again by using FACS washing buffer and treated with anti-human IgG, which was conjugated with FITC (fluorescein isothiocyanate) a fluorescent dying agent. In addition, the reaction solution was maintained at 4(C for 40 minutes and washed once more by using the FACS washing solution.

Step 2: Flow Cytometry

In order to perform the measurement of binding strength between the prepared cells and antibodies in each sample, a FACSort instrument (Becton Dickinson) was pre-started and the

‘Cell Quest’ program (trademark) was used. Then, a dot plot was established and for the plot panel the plot source was indicated in the acquisition state. Besides, in option cells were determined to be calculated to only 10,000 events. For the histogram plot, the histogram source was indicated in the acquisition state and the first parameter was decided to be FL1-H 500. Again, for the histogram plot, the peak of DHFR negative CHO cell was controlled to be in the range of 100 101. In the dot plot, the FCS axis and SSC axis were placed in the linear mode and during the analysis process, FSC values were controlled deliberately before and after 500. Then, the cells with large SSC values were calculated to obtain data, namely live cells were selected by gating in case that dead cells had a high portion. Once the peak of the negative control group was determined, the samples treated with antibodies 4B4-1-1 and LB00506 were added to be measured. At that time, the X axis in the histogram plot indicated the fluorescence as FL1 and the Y-axis indicated the number of cells corresponding to the above values. As described above, the binding affinity between the humanized antibody LB00606 and cell surface 4-1BB molecule was estimated by using a FACS instrument for the analysis. As a result, it was confirmed that the binding strength hardly changed, compared with that before humanization (See FIG. 12), which meant that the binding affinity to activated T cells was scarcely affected during humanization.

### EXAMPLE 15

### Measurement of Immuno-Suppression of the Humanized Antibody LB00506

Step 1: Preparation of PBMC

Blood treated with sodium citrate (sodium citrate:blood 1:9) was diluted with PBS buffer in the same ratio. Then Ficoll-Hypaque solution (Sigma) was poured into a 50 ml conical tube in the same volume of the above blood diluted with PBS buffer first and the resulting blood was added slowly. Second, the mixture was centrifuged for 30 minutes at 2,000 rpm (900 * g) and collected in a newly sterilized tube. Then the cells were washed twice and resuspended to a concentration of 1×10⁶ cells/ml by using complete RPMI medium.

Step 2: Reaction of Standard One Way MLR

PBMC, which can be used for stimulating cells, was treated with mitomycin C. At that time, the final concentration of mitomycin C was adjusted to 25 (g/ml and cells were cultivated at 37(C for 30 minutes. Then the resulting cells were washed 3 times. PBMC which can be utilized for reacting cells was prepared to a concentration of 1×10⁶ cells/ml by using complete RPMI medium. Then, the stimulating cells and the reacting cell were mixed and put into a 96-well plate with a U bottom with 50 (μl per well. Then, the antibody of the present invention was diluted successively to become concentrations of 0.1 (g/(l, 1 (g/(l, 10 (g/(l and added at 100 (1 per well. At that time, for the negative control group, human IgG was adopted. As prepared above, the microplate was incubated at 37(C for 72 hours and 50 (Ci/ml per well of 3H-thymidine (Amersham) was added in 20 (μl amount and were cultivated for more than 16 hours. The cultivated cells were harvested by using a 96-well cell harvester (Packard) and Unifilter-96 GF/B filter plate (Packard) and the remnant ³H-thymidine that remained within the supernatant was removed. Then, the Unifilter was dried in the dryer and attached with adhesion tape for sealing its bottom surface so as to be added with scintillation fluid in 35 (1 per well. Then, the Unifilter was sealed by using TopSealTM-S (trademark) and measured with a microplate scintillation counter (trademark TopCount; Packard).

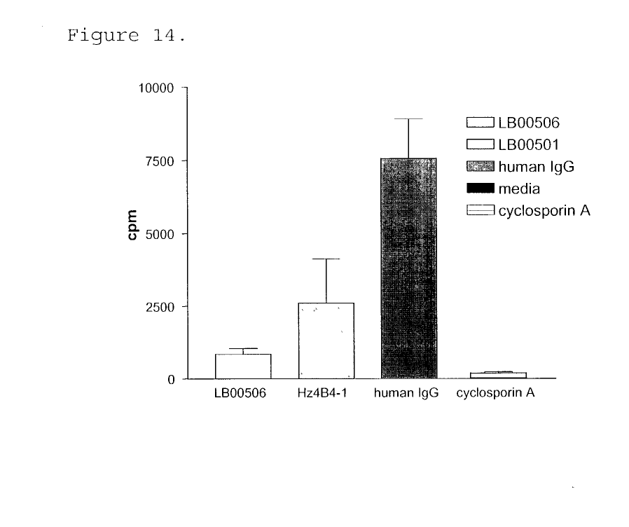

In accordance with the procedures described above, each antibody was examined to have an inhibitory effect at a 1 (μl/ml of fixed concentration. As a result, the immuno-suppression was calculated to be 87.6% in the humanized antibody LB00506, 62% in the humanized antibody LB00501 and 0% in IgG. Therefore, it was confirmed that the antibody LB00506 had a higher inhibitory effect than that of the antibody LB00501. In addition, the immuno-suppression of the antibody LB00506 was specific for T cells expressing the 4-1BB molecule since human IgG utilized for the negative control group showed no activity. Besides, although the antibody LB00506 had a lower immuno-suppression than cyclosporin A apparently, the inhibitory activity of antibody LB00506 in 10 (μl/ml of concentration was identified to have no difference statistically compared with that of cyclosporin A. Then, although cyclosporin A showed excellent efficacy for immuno-suppression, it affected various organs non-specifically so as to induce kidney toxicity and immune toxicity. Therefore, the selective immuno-suppression of antibody LB00506 was more useful and meaningful since it worked only T cells selectively (See FIG. 14).

### EXAMPLE 16

### Experimental Results—Immune Response to and Immunosuppressive Effect of the Humanized Antibody

A. Immune Response—in Baboon and Administration of the Humanized Antibody Hz4B4-1

Seven- to eight-year-old male and female baboons (P. anubis), weighing 12 to 15 kg, at the Southwest Foundation for Biomedical Research (San Antonio, Tex.), were used. The baboons were immunized intramuscularly with 1 mg of OVA (Sigma) emulsified in aluminum hydroxide (Sigma). The time of OVA-immunization was designated as week 0, and OVA (1 mg in PBS) was given again at week 6. The first injection of humanized antibody (or PBS, control group) was given at the time of the first OVA immunization (we group) was injected intravenously with 10 ml PBS. The second and third groups were treated with the humanized antibody Hz4B4-1 obta week 10.

B. Evaluation of Host Humoral Immune Response

Anti-OVA and IgM levels were determined by ELISA. Immunomaxisorp™ plates (Nunc InterMed, Rockilde, Denmark) were coated with OVA at a concentration of 500 ng/well. After blocking with 1% bovine serum albumin, serial twofold dilutions of ser room temperature. Bound IgG and IgM were detected with alkaline phosphatase (AP)-conjugated rabbit anti-monkey IgG (Sigma) or AP-conjugated goat anti-human IgM (Sigma), respectively by adding 0.1 ml of the conjugates to each well and incubating at the above temperature. A₄₀₅ was read by an automa absorbance three or five times the background reading at A₄₀₅, respectively.

Total IgG or Hz4B4-1 in the sera was quantified by ELISA. Diluted serum samples were added as above to the plates coated with goat anti-human IgG (150 ng/well) or GST-4-1BB (100 ng/well) and incubated for 4 hours at the above indicated temperature. After incubation, AP-conjugated goat anti-human IgG (Sigma) was added to each well of the plates and development of the reaction was performed using p-nitrophenyl phosphat each sample was calculated with reference to standard curves generated by using human IgG or purified Hz4B4-1, respectively.

TABLE 4

| | | Ratio of | Ratio of | | |
|---|---|---|---|---|---|
| | | OVA- | OVA- | | |
| | | specific | specific | | |
| | | IgG titer | IgM titer | Ratio of | |
| | | at wk 7 | at wk 7 | serum cone. | |
| | | to OVA- | to OVA- | of total IgG | Serum |
| | | specific | specific | at wk 7 to | cone. of |
| Exp. | Dose of | IgG | IgM | serum cone. | Hz4B4-1 |
| Animal | Hz4B4-1 | titer at | titer at | of total IgG | at wk 1 |
| No. | (mg/kg) | wk 0 | wk 0 | at wk 1 | (μg/ml) |
| A1 | 0 | 64 | 2 | 1.13 | — |
| A2 | 0 | 16 | 1 | 1.02 | — |
| A3 | 0 | 32 | 1 | 1.05 | — |
| B1 | 1 | 2 | 1 | 0.96 | 6.66 |
| B2 | 1 | 128 | 1 | 1.06 | 0.42 |
| B3 | 1 | 2 | 1 | 1.01 | 3.85 |
| C1 | 4 | 4 | 2 | 1.09 | 25.74 |
| C2 | 4 | 1 | 0.5 | 1.16 | 10.14 |
| C3 | 4 | 2 | 1 | 1.09 | 14.49 |

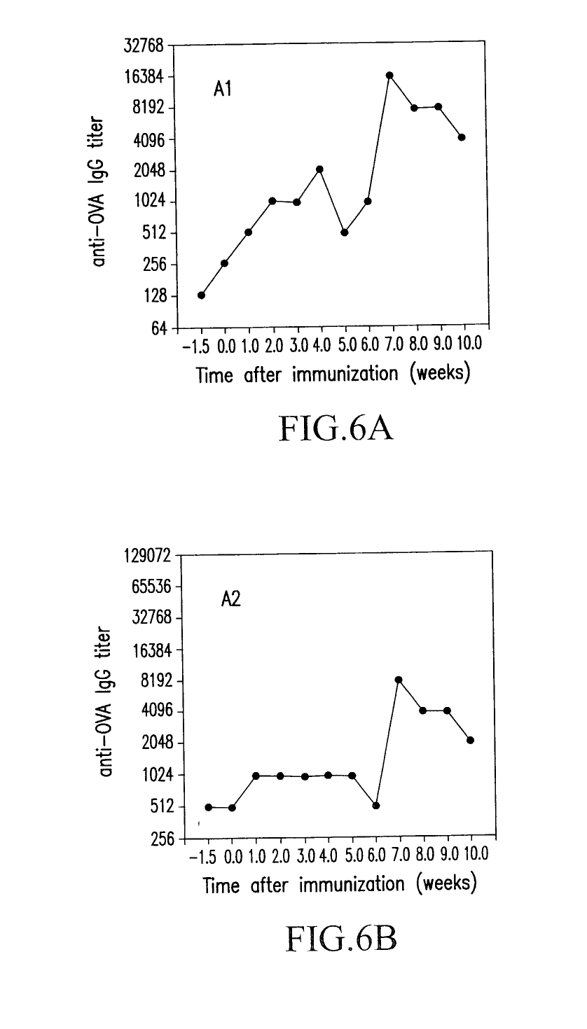

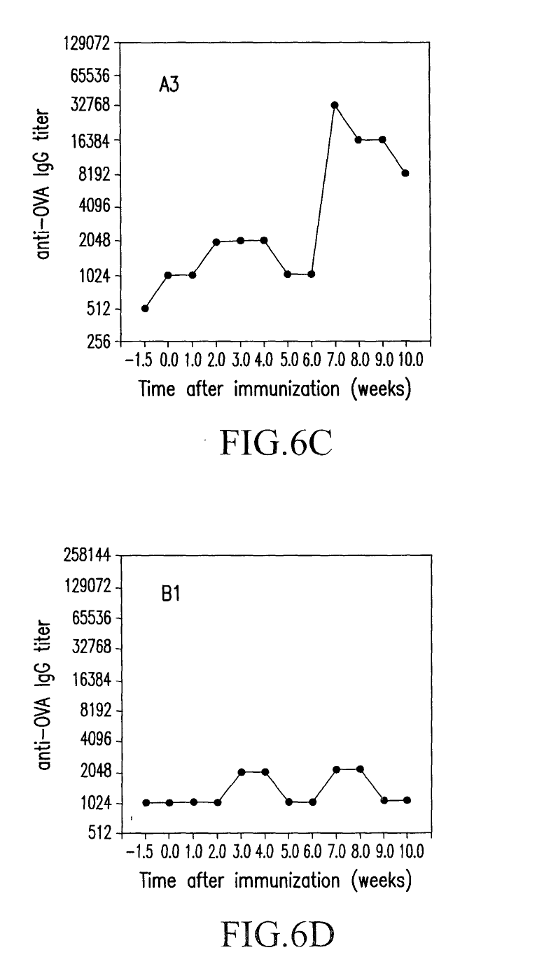

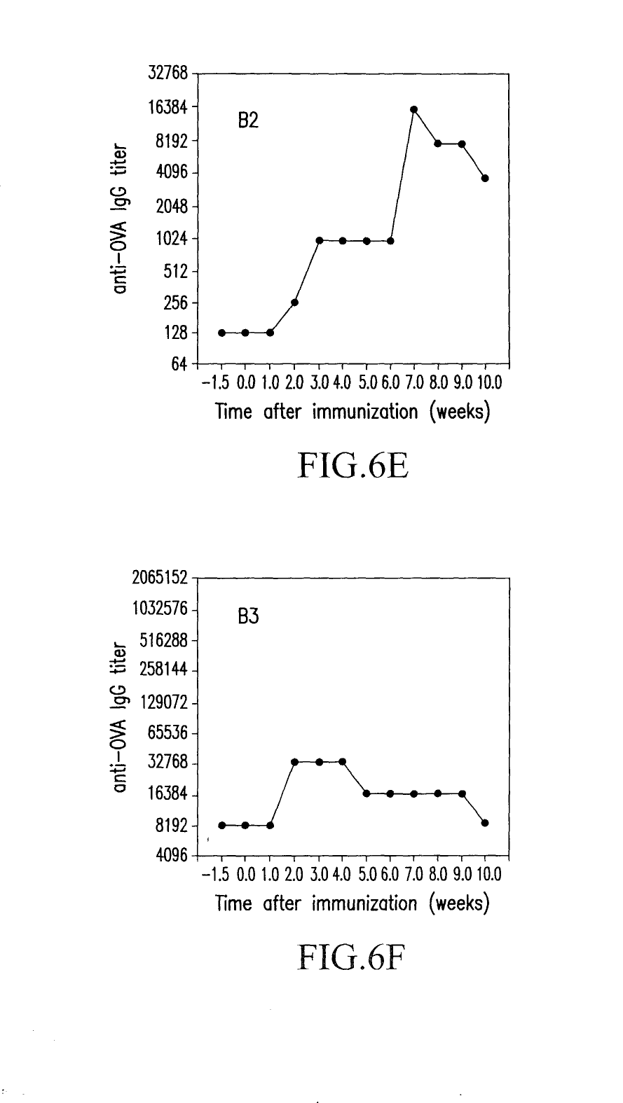

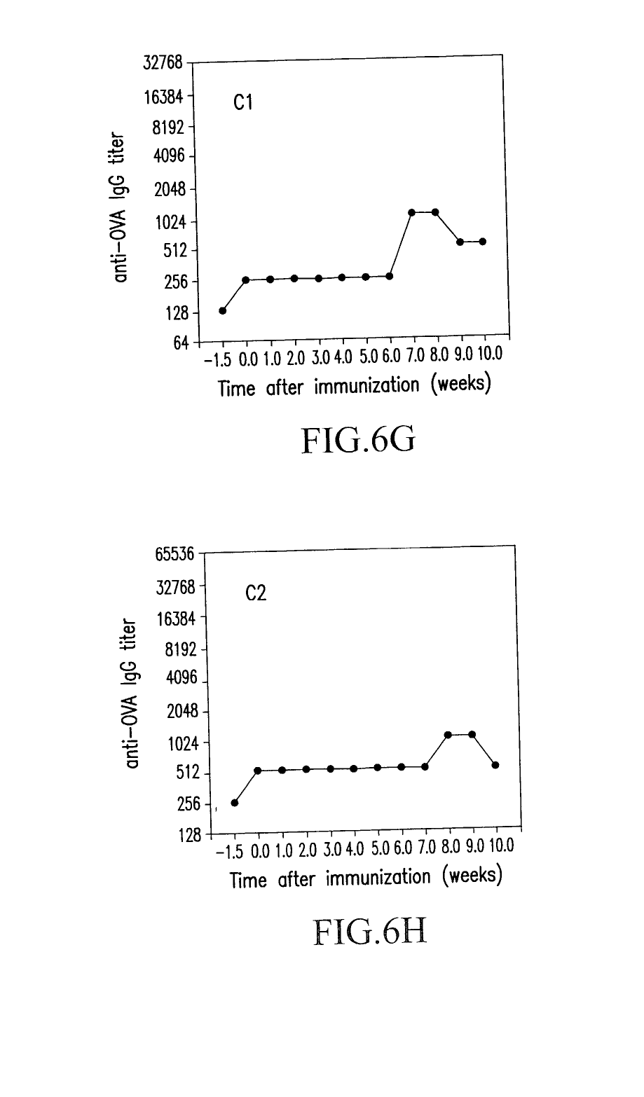

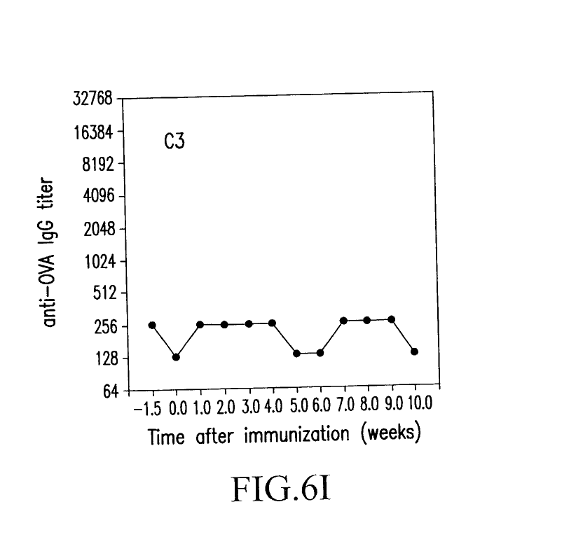

A distinguishable increase of the anti-OVA IgG level in the serum was found in the control group. The highest titers were observed at week 7, a week after the second immunization with OVA, and were 64-, 16-, and 32-fold greater than the titer at week 0 in baboons A1, A2, and A3, respectively (Table 4 and FIGS. 6a, 6b, and 6c). In contrast, significant suppression of the OVA-specific Ab response was found in Hz4B4-1-treated baboons. In the second group, which was treated with a dose of 1 mg/kg of Hz4B4-1, two of three baboons (B1 and B3) showed the suppression. The titers at week 7 were twofold higher than those at week 0 (Table 1, and FIGS. 6d, 6e, and 6f). However, the suppression was not detected in baboon B2, which showed a 128-fold increase in titer between week 7 and week 0. In the case of the third group, which was treated with 4 mg/kg of Hz4B4-1, the suppression was shown in all three baboons. The titer increase was four-, one-, and two-fold for baboons C1, C2, and C3, respectively (Table 1, and FIGS. 6g, 6h, and 6i).

No distinguishable increase in anti-OVA IgM titer was observed in any of the animals, irrespective of whether they were treated with Hz4B4-1, indicating that Hz4B4-1 treatment did not affect the IgM production at a detectable level. Taken together, the data suggest that a state of humoral unresponsiveness to OVA, a T cell-dependent antigen, was induced by Hz4B4-1 treatment. This was further indicated by measuring the serum concentration of Hz4B4-1. The serum concentration of Hz4B4-1 was highest at week 1 in all baboons. The concentration of Hz4B4-1 in the serum of baboon B2, which did not show OVA-specific IgG suppression, was significantly

lower compared with those of baboons B1 and B3 treated with the same dose of Hz4B4-1 (Table 1). The amount of total

IgG at week 0 was compared with that of week 7 in each baboon. As shown in Table 1, the total IgG amount was little altered, regardless of the treatment with Hz4B4-1. In addition, during the treatment with Hz4B4-1, no significant

variations were observed in total numbers and proportions of B and T cells in each blood sample analyzed by flow cytometry. Collectively, the data indicated that the immune unresponsiveness by treatment with Hz4B4-1 was Ag-specific, and not due to overall immune suppression.

C. Analysis of 4-1BB-Positive T-Cells

To evaluate the clinical significance of 4-1BB molecules, the expression of these molecules on T lymphocytes of patients with rheumatoid arthritis (RA) was analyzed by FRCS analysis using 4B4 MAb. PBMC was prepared from heparinized venous blood withdrawn from normal volunteers or RA patients, and synovial cells were obtained from synovial fluid aspirated from RA patients. 5×10⁵ PBMC or synovial cells were incubated with 5 μl of phycoerythrin (PE)-labeled anti-human CD4 or PE-labeled anti-human CD8 mouse MAb (Immune Source, Los Altos, Calif.) in a staining buffer that contains DMEM (1% BSA and 0.005% NaNH₃) for 30 min at 4° C. in the dark. After incubation, cells were washed with staining buffer, and then incubated with 5 μl of FITC-labeled 4B4 MAb for 30 min at 4° C. in staining buffer. The percentage of 4-1BB expressing T lymphocytes was analyzed by flow cytometry. The expression of 4-1BB molecules on T lymphocytes was analyzed on a FACStar Plus™ cytometer (Becton Dickinson & Co., Mountain View, Calif.).

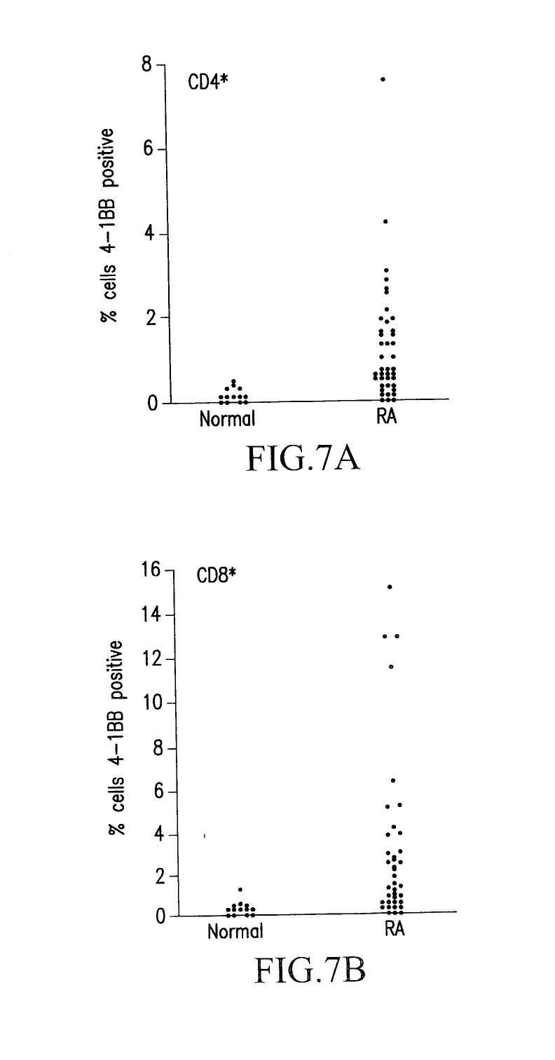

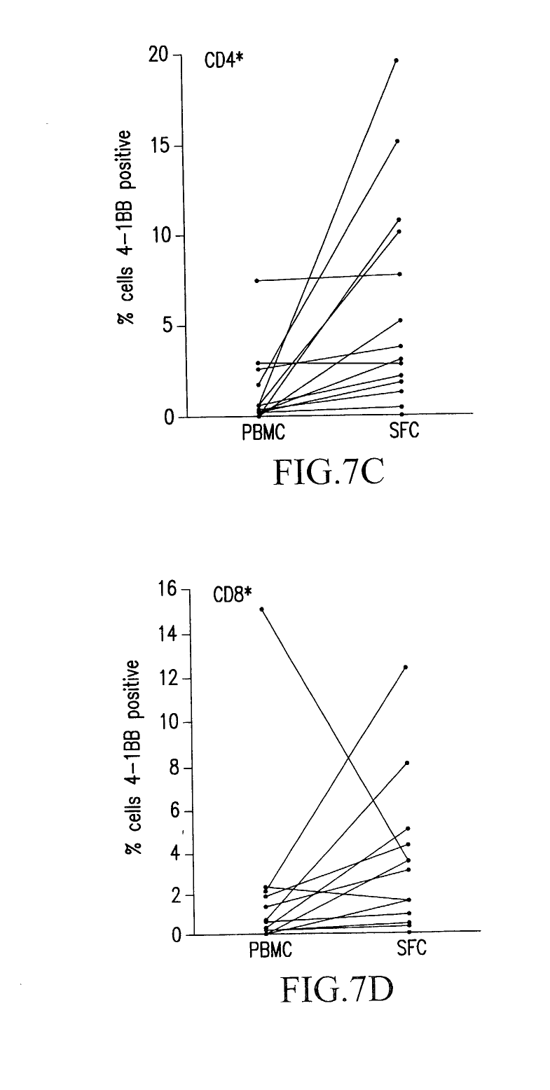

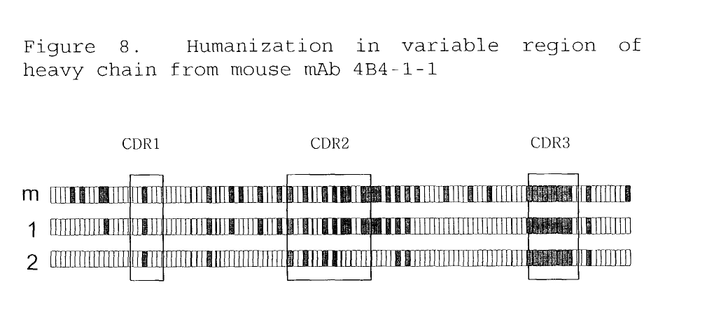

The expression of these molecules on peripheral blood (PBMC) T lymphocytes of 41 RA patients was compared with those of 13 normal individuals. The results are shown in FIGS. 7a, and 7b . The reactivity with 4B4 MAb of CD4 or CD8 T lymphocytes of normal individuals was very low or not observed: CD4⁺, <0.5%; CD8⁺, <1,2%. In contrast, the CD4⁺ or CD8⁺ T lymphocytes from some of the RA patients showed the increased reactivity to 4B4 MAb. In the case of CD4⁺ T cells, the reaction rates were above 1% in 18 of 41 patients and peaked at 7.5%. In CD8⁺ T cells, 17 of 38 patients showed reaction rates above 2%, with a maximum value of 15%.

RA is characterized by chronic synovitis. Affected synovial tissues are infiltrated with lymphocytes and plasma cells. This disease is initiated by activation of T lymphocytes responding to some arthritogenic agents, and T lymphocytes play a primary role in the pathogenesis of RA, indicating that T lymphocytes in synovial fluid of RA patients are in an activated state (G. S. Firestein, p. 851 in “Etiology and Pathogenesis of Rheumatoid Arthritis”, 5th ed., W. N. Kelley et al. eds., c. 1997 by W. B. Saunders, Philadelphia, Pa.). Therefore, the expression of 4-1BB molecules in synovial fluid T lymphocytes of 13 RA patients were compared with those in the peripheral blood T lymphocytes of the same patients. The results are shown in FIGS. 7c and 7d. CD4⁺ and CD8⁺ T lymphocytes in synovial fluid showed more reactivity to 4B4 MAb than those in peripheral blood; for the CD4⁺ or CD8⁺ subset, above twofold in 8 or 9 RA patients out of 13. Each line between two circles means that the corresponding PBMC and SFC were obtained from the same patient. These findings suggest that the expression of 4-1BB may be related to the disease process of RA.

Without being bound by any theory of the invention, the inventors speculate that there are two not mutually exclusive mechanisms of the immunosuppressive effect of the Hz4B4-1. First, the antibody, could interfere with 4-1BB/4 1BBL interaction that plays an important role in T cell activation. Second, the antibody could eliminate 4-1BB expressing T cells via complement-dependent cytotoxicity and antibody-dependent cellular toxicity. In many cases of T cell mediated autoimmune diseases, autoantigens are not well defined or are too diverse to manipulate the immune response against the autoantigens. In this regard, functional blocking and/or elimination of activated T cells, most of which are probably autoantigen specific in patients with autoimmune diseases, could be approaches to ameliorate the disease. The inventors have discovered that a substantial proportion of T cells in PBMC and synovial fluid from RA patients express 4-1BB, which suggests that 4-1BB could be an ideal target for antibody mediated therapy for RA since only activated T cells, possibly most pathologic T cells, express 4-1BB. In this regard, a longer expression time of 4-1BB (more than 72 hours) than those of other costimulatory molecules such as CD40L, may be another advantageous point for targeting 4-1BB with the antibody. Beyond RA, Hz4B4-1 could also be used for the treatment of other T cell mediated autoimmune diseases and graft rejections.

The present specification includes the appended Sequence Listing of 49 nucleic acid or amino acid sequences. Articles of the patent and scientific periodical literature cited herein are hereby incorporated in their entirety by such citation.

### INDUSTRIAL APPLICABILITY

The present invention relates to humanized monoclonal antibodies LB-00503 and LB-00506, which are specific for human 4-1BB molecule, have high binding affinities, can bind toward activated T cells expressing the 4-1BB molecule and are similar to human antibodies structurally as well as their pharmaceutical compositions.

Those skilled in the art will appreciate that the conceptions and specific embodiments disclosed in the foregoing description may be readily utilized as a basis for modifying or designing other embodiments for carrying out the same purposes of the present invention.

Those skilled in the art will also appreciate that such equivalent embodiments do not depart from the spirit and scope of the invention as set forth in the appended claims.

We claim:

1. A humanized antibody against a human 4-1BB molecule comprising:
a) an antibody light chain having characteristic amino acid sequences for complementarity determining regions in an antigen binding region denoted by,
RSAQTIXDYLH YASQSIS QDGHSFPPT
wherein X is any amino acid,
b) an antibody heavy chain having characteristic amino acid sequences for complementarity determining regions in the antigen binding region denoted by,
SYWMH EINPGNGHTNYXXKFXX SFTTARAFAY
X is any amino acid,
d) a light chain constant region identical to a human antibody light chain constant region; and
e) a heavy chain constant region identical to a human antibody heavy chain constant region.

2. The humanized antibody of claim 1, wherein up to ten amino acids in framework portion of the light chain are substituted by corresponding amino acids from a mouse monoclonal antibody that specifically binds to h4-1BB protein.

3. The humanized antibody of claim 1, wherein up to ten amino acids in framework portion of the heavy chain are substituted by corresponding amino acids from a mouse monoclonal antibody that specifically binds to h4-1BB protein.

4. The humanized antibody of claim 1, wherein a K_{d} of said humanized antibody for h4-1BB is less than or equal to 8.7×10⁻¹¹M.

5. A humanized antibody comprising:
a) a light chain variable region comprising a polypeptide having the amino acid sequence of SEQ ID NO.5:
b) a heavy chain variable region comprising a polypeptide having the amino acid sequence of SEQ ID NO.6:
c) a light chain constant region identical to a human antibody light chain constant region; and
d) a heavy chain constant region identical to a human antibody heavy chain constant region,
wherein said humanized antibody is expressed from cells of the cell line deposited as KCTC 10165BP.

6. The humanized antibody of claim 5 wherein the heavy chain is a gamma 1, gamma 2, gamma 3 or gamma 4 chain.

7. The humanized antibody of claim 5 wherein the light chain is a kappa or lambda light chain.

8. A plasmid comprising a polynucleotide encoding a light chain variable region of a humanized antibody, wherein said plasmid is pRc-Hz4B4-k-gs or pRc-Hz4B4-Mok-gs resident in cells deposited as KCTC 0537BP.

9. A plasmid comprising a polynucleotide encoding a heavy chain variable region of a humanized antibody, wherein said plasmid is pCI-Hz4B4-H or pCI-Hz4B4-MoH resident in cells deposited as KCTC 0536BP.

10. A plasmid comprising a polynucleotide encoding a heavy chain and a light chain variable region of a humanized antibody, wherein said plasmid is pANTIBODY-VER6 resident in cells deposited as KCTC 10165BP.

11. A host cell transformed with the plasmid of claim 8.

12. A host cell transformed with the plasmid of claim 9.

13. A host cell transformed with the plasmid of claim 10.

14. A cell of a cell line deposited as KCTC 0540BP, KCTC 0541BP or as KCTC 10165BP.

15. A pharmaceutical composition for treating autoimmune disease and suppressing immune reactions comprising a humanized antibody and one or more pharmaceutically acceptable substances.

16. The pharmaceutical composition of claim 15, wherein said humanized antibody is an antibody of any one of claim 1-7.

17. The pharmaceutical composition according to claim 15, wherein said humanized antibody comprises;
a) a light chain variable region comprising a polypeptide having the amino acid sequence of SEQ ID NO.1:
b) a heavy chain variable region comprising a polypeptide having the amino acid sequence of SEQ ID NO.2:
c) a light chain constant region identical to a human antibody light chain constant region; and
d) a heavy chain constant region identical to a human antibody heavy chain constant region,
wherein said humanized antibody is expressed from cells of the cell line deposited as KCTC 0540BP.

18. The pharmaceutical composition of claim 15, wherein said humanized antibody comprises;
a) a light chain variable region comprising a polypeptide having the amino acid sequence of SEQ ID NO.3:
b) a heavy chain variable region comprising a polypeptide having the amino acid sequence of SEQ ID NO.4:
c) a light chain constant region identical to a human antibody light chain constant region; and
d) a heavy chain constant region identical to a human antibody heavy chain constant region,
wherein said humanized antibody is expressed from cells of the cell line deposited as KCTC 0541BP.

19. The pharmaceutical composition of 15, wherein said autoimmune disease is rheumatoid arthritis.

20. A method for suppressing an immune response in a patient comprising administering the composition of any one of claims 15-19 to said patient in an amount effective to suppress said immune response.

21. A method for detecting presence of an autoimmune response in a patient in vitro comprising:
contacting a sample from said patient with an antibody that specifically binds to H4-1BB protein, and
determining the number of CD4⁺ and CD8⁺ cells that are specifically bound by said antibody,
wherein an autoimmune response is present if greater than 1% of said CD4⁺ cells or greater than 2% of said CD8⁺ cells are labeled.

22. The method of claim 21, wherein said antibody is an antibody of any one of claims 1-7.

23. A manufacturing method for a humanized antibody wherein specificity for a human 4-1BB molecule is enhanced, comprising modifying 11 or fewer amino acid residues in variable region of humanized antibody Hz4B4-2 which specifically binds a human 4-1BB molecule, wherein the modifying includes substitution and/or reduction (or restoration) of a glycosylation site in-the variable region of the humanized antibody Hz4B4-2.

24. The manufacturing method of claim 23, wherein modifying the amino acid residues is carried out by site-directed mutagenesis using an expression vector having the nucleotide sequence of humanized antibody Hz4B4-2 with binding specificity for the human 4-1BB molecule as a template, wherein expression of the humanized antibody with the expression vector is in a cell line system.

25. The manufacturing method of claim 24, wherein the site-directed mutagenesis includes any of the following;
(1) substitution of serine at the 61st amino acid position, in the 59th˜61st amino acid residues of CDR2 in the region of variable heavy chain, by one or more asparagines;
(2) substitution of the 65^{th} and 66^{th} amino acid residues, glutamine-glycine, situated at C-terminus of the antigen binding site in the variable region of heavy chain, by lysine-serine; or
(3) both (1) and (2) at the same time.

26. The manufacturing method of claim 24, wherein expression is carried out by using a mammalian cell system.
